(19) United States
(12) Patent Application Publication
(10) Pub. No.: US 2012/0157482 A1
(43) Pub. Date: Jun. 21, 2012

(54) COMPOUNDS AND METHODS

(76) Inventors: Alan Peterson Graves, III, King of Prussia, PA (US); Lara S. Kallander, King of Prussia, PA (US); Patrick Stoy, King of Prussia, PA (US)

(21) Appl. No.: 13/392,904

(22) PCT Filed: Aug. 25, 2010
(86) PCT No.: PCT/US10/46564

§ 371 Date: Feb. 28, 2012

Related U.S. Application Data
(60) Provisional application No. 61/237,815, filed on Aug. 28, 2009.

(51) Int. Cl. A61K 31/505; C12N 9/99; A61P 9/04; C07D 239/47

(52) U.S. Cl. 514/269; 544/319; 435/184

(57) ABSTRACT

Disclosed are compounds having the formula:

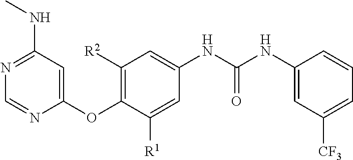

wherein R¹ and R² are as defined herein, and methods of making and using the same.

### FIELD OF THE INVENTION

The present invention relates to compounds that inhibit TNNI3K and methods of making and using the same. Specifically, the present invention relates to N-(4-{[6-(methylamino)-4-pyrimidinyl]oxy}phenyl)-N′-[3-(trifluoromethyl)phenyl]ureas as TNNI3K inhibitors.

### BACKGROUND OF THE INVENTION

Cardiac troponin I-interacting kinase (TNNI3K), also known as CARK (for cardiac ankyrin repeat kinase), is a protein kinase that exhibits highly selective expression for cardiac tissues and has been shown to interact with components of the sarcomere, including troponin I (Zhao, Y. et al., J. Mol. Med., 2003, 81, 297-304; Feng, Y. et al., Gen. Physiol. Biophys., 2007, 26, 104-109; Wang, H. et al., J. Cell. Mol. Med., 2008, 12, 304-315). Although substrates for TNNI3K have not been identified to date, recent reports suggest that this protein may play a role in the development of pressure-induced cardiomyocyte hypertrophy and contractile dysfunction (Wheeler, F. C. et al., Mamm. Genome, 2005, 16, 414-423; Wang, X. et al. “TNNI3K, a cardiac-specific kinase, promotes cardiac hypertrophy in vivo”, Poster presentation at the 2006 Scientific Sessions of the American Heart Association, Chicago, Ill.). Inhibition of the kinase activity of TNNI3K may disrupt these signaling pathways, and enable the mitigation and/or reversal of cardiac hypertrophy seen in patients with progressively worsening heart failure.

In response to mechanical, neurohormonal, and genetic stimuli, the heart will undergo hypertrophy, or muscle growth and remodeling, in order to maintain sufficient cardiac output to meet tissue oxygen demands. While these structural changes are initially seen as compensatory, sustained dysregulation of hypertrophic signaling can lead to heart failure, the pathophysiogical state in which the heart can no longer adequately function as a pump (Mudd, J. O. and Kass, D. A., Nature, 2008, 451, 919-928). Prevention or reversal of pathological cardiac hypertrophy has the potential to delay or prevent the development of congestive heart failure (McKinsey, T. A. and Kass, D. A., Nat. Rev. Drug Discov., 2007, 6, 617-635; Kaye, D. M. and Krum, H., Nat. Rev. Drug Discov., 2007, 6, 127-139).

Heart failure is responsible for a reduced quality of life and premature death in a significant proportion of sufferers, and is characterized by impaired cardiac function either due to reduced pump function (systolic dysfunction) or reduced filling (diastolic dysfunction). Congestive heart failure (CHF) is characterized by impaired left ventricular function, increased peripheral and pulmonary vascular resistance and reduced exercise tolerance and dyspnea. The prevalence of heart failure is anticipated to increase with ageing populations, prompting a need for new and improved methods of treating heart failure.

### SUMMARY OF THE INVENTION

The invention is directed to novel di-aryl ureas. Specifically, the invention is directed to compounds according to Formula I or a salt thereof:

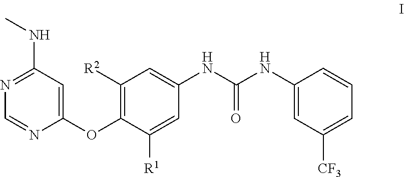

wherein:
R¹ is halogen, C₁-C₄ alkyl, or —ORₐ;
R² is H, halogen, C₁-C₄ alkyl, or —ORₐ; and
Rₐ is C₁-C₄ alkyl which is optionally substituted one to three times by halogen;
provided that when R² is H, R¹ is not methyl.

The compounds of the invention are inhibitors of TNNI3K and can be useful for the treatment of cardiac diseases and disorders, particularly heart failure. Accordingly, the invention is further directed to pharmaceutical compositions comprising a compound of the invention. The invention is still further directed to methods of inhibiting TNNI3K and treatment of conditions associated therewith using a compound of the invention or a pharmaceutical composition comprising a compound of the invention.

### DETAILED DESCRIPTION OF THE INVENTION

As used herein, the term “alkyl” represents a straight- or branched-chain saturated hydrocarbon, which may be unsubstituted or substituted by one, or more of the substituents defined herein. Exemplary alkyls include, but are not limited to methyl (Me), ethyl (Et), propyl, isopropyl, butyl, isobutyl, and t-butyl. The term “C₁-C₄ alkyl” refers to an alkyl group containing from 1 to 4 carbon atoms.

The terms “halogen” and “halo” represent chloro, fluoro, bromo or iodo substituents.

One embodiment of the invention is a compound of Formula I or a pharmaceutically acceptable salt thereof wherein R¹ is selected from chloro, fluoro, bromo, methyl, ethyl, methoxy, and trifluoromethoxy; and R² is selected from hydrogen, chloro, fluoro, bromo, and methyl. In another embodiment of the invention R² is selected from chloro, fluoro, bromo, and methyl.

Specific compounds of this invention are:
N-(3,5-dichloro-4-{[6-(methylamino)-4-pyrimidinyl]oxy}phenyl)-N′-[3-(trifluoromethyl)phenyl]urea,
N-(3,5-dibromo-4-{[6,-(methylamino)-4-pyrimidinyl]oxy}phenyl)-N′-[3-(trifluoromethyl)phenyl]urea,
N-(3-bromo-5-chloro-4-{[6-(methylamino)-4-pyrimidinyl]oxy}phenyl)-N′-[3-(trifluoromethyl)phenyl]urea,
N-(3,5-difluoro-4-{[6-(methylamino)-4-pyrimidinyl]oxy}phenyl)-N′-[3-(trifluoromethyl)phenyl]urea,
N-(3,5-dimethyl-4-{[6-(methylamino)-4-pyrimidinyl]oxy}phenyl)-N′-[3-(trifluoromethyl)phenyl]urea,
N-[3-bromo-4-{[6-(methylamino)-4-pyrimidinyl]oxy}-5-(methyloxy)phenyl]-N′-[3-(trifluoromethyl)phenyl]urea,
N-(3-chloro-4-{[6-(methylamino)-4-pyrimidinyl]oxy}phenyl)-M-[3-(trifluoromethyl)phenyl]urea,
N-(3-bromo-4-{[6-(methylamino)-4-pyrimidinyl]oxy}phenyl)-N′-[3-(trifluoromethyl)phenyl]urea,
N-[4-{[6-(methylamino)-4-pyrimidinyl]oxy}-3-(methyloxy)phenyl]N′[3-(trifluoromethyl)phenyl]urea,
N-[3-chloro-4-{[6-(methylamino)-4-pyrimidinyl]oxy}-5-(methyloxy)phenyl]-N′-[3-(trifluoromethyl)phenyl]urea,
N-(3-fluoro-4-{[6-(methylamino)-4-pyrimidinyl]oxy}phenyl)-N′-[3-(trifluoromethyl)phenyl]urea,
N-(3-chloro-5-methyl-4-{[6-(methylamino)-4-pyrimidinyl]oxy}phenyl)-N′-[3-(trifluoromethyl)phenyl]urea,
N-(3-chloro-5-ethyl-4-{[6-(methylamino)-4-pyrimidinyl]oxy}phenyl)-N′-[3-(trifluoromethyl)phenyl]urea, and
N-{3-chloro-4-{[6-(methylamino)-4-pyrimidinyl]oxy}-5-[(trifluoromethyl)oxy]phenyl}-N′-[3-(trifluoromethyl)phenyl]urea,
and salts, particularly pharmaceutically acceptable salts, thereof.

Representative compounds of this invention include the compounds of Examples 1-14.

The compounds according to Formula I may contain one or more asymmetric center (also referred to as a chiral center) and may, therefore, exist as individual enantiomers, diastereomers, or other stereoisomeric forms, or as mixtures thereof. Chiral centers, such as chiral carbon atoms, may also be present in a substituent such as an alkyl group. Where the stereochemistry of a chiral center present in Formula I, or in any chemical structure illustrated herein, is not specified the structure is intended to encompass all individual stereoisomers and all mixtures thereof. Thus, compounds according to Formula I containing one or more chiral center may be used as racemic mixtures, enantiomerically enriched mixtures, or as enantiomerically pure individual stereoisomers.

Individual stereoisomers of a compound according to Formula I which contain one or more asymmetric center may be resolved by methods known to those skilled in the art. For example, such resolution may be carried out (1) by formation of diastereoisomeric salts, complexes or other derivatives; (2) by selective reaction with a stereoisomer-specific reagent, for example by enzymatic oxidation or reduction; or (3) by gas-liquid or liquid chromatography in a chiral environment, for example, on a chiral support such as silica with a bound chiral ligand or in the presence of a chiral solvent. The skilled artisan will appreciate that where the desired stereoisomer is converted into another chemical entity by one of the separation procedures described above, a further step is required to liberate the desired form. Alternatively, specific stereoisomers may be synthesized by asymmetric synthesis using optically active reagents, substrates, catalysts or solvents, or by converting one enantiomer to the other by asymmetric transformation.

When a disclosed compound or its salt is named or depicted by structure, it is to be understood that the compound or salt, including solvates (particularly, hydrates) thereof, may exist in crystalline forms, non-crystalline forms or a mixture thereof. The compound or salt, or solvates (particularly, hydrates) thereof, may also exhibit polymorphism (i.e. the capacity to occur in different crystalline forms). These different crystalline forms are typically known as “polymorphs.” It is to be understood that when named or depicted by structure, the disclosed compound, or solvates (particularly, hydrates) thereof, also include all polymorphs thereof. Polymorphs have the same chemical composition but differ in packing, geometrical arrangement, and other descriptive properties of the crystalline solid state. Polymorphs, therefore, may have different physical properties such as shape, density, hardness, deformability, stability, and dissolution properties. Polymorphs typically exhibit different melting points, IR spectra, and X-ray powder diffraction patterns, which may be used for identification. One of ordinary skill in the art will appreciate that different polymorphs may be produced, for example, by changing or adjusting the conditions used in crystallizing/recrystallizing the compound.

For solvates of the compounds of the invention, or salts thereof, that are in crystalline form, the skilled artisan will appreciate that pharmaceutically acceptable solvates may be formed wherein solvent molecules are incorporated into the crystalline lattice during crystallization. Solvates may involve nonaqueous solvents such as ethanol, isopropanol, DMSO, acetic acid, ethanolamine, and ethyl acetate, or they may involve water as the solvent that is incorporated into the crystalline lattice. Solvates wherein water is the solvent that is incorporated into the crystalline lattice are typically referred to as “hydrates.” Hydrates include stoichiometric hydrates as well as compositions containing variable amounts of water. The invention includes all such solvates.

Because of their potential use in medicine, the salts of the compounds of Formula I are preferably pharmaceutically acceptable. The compounds of this invention are bases, wherein a desired salt form may be prepared by any suitable method known in the art, including treatment of the free base with an inorganic acid, such as hydrochloric acid, hydrobromic acid, sulfuric acid, nitric acid, phosphoric acid, and the like, or with an organic acid, such as acetic acid, trifluoroacetic acid, maleic acid, succinic acid, mandelic acid, fumaric acid, malonic acid, pyruvic acid, oxalic acid, glycolic acid, salicylic acid, pyranosidyl acid, such as glucuronic acid or galacturonic acid, alpha-hydroxy acid, such as citric acid or tartaric acid, amino acid, such as aspartic acid or glutamic acid, aromatic acid, such as benzoic acid or cinnamic acid, sulfonic acid, such as p-toluenesulfonic acid, methanesulfonic acid, ethanesulfonic acid or the like. Examples of pharmaceutically acceptable salts include sulfates, pyrosulfates, bisulfates, sulfites, bisulfites, phosphates, chlorides, bromides, iodides, acetates, propionates, decanoates, caprylates, acrylates, formates, isobutyrates, caproates, heptanoates, propiolates, oxalates, malonates succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, phenylacetates, phenylpropionates, phenylbutrates, citrates, lactates, γ-hydroxybutyrates, glycollates, tartrates mandelates, and sulfonates, such as xylenesulfonates, methanesulfonates, propanesulfonates, naphthalene-1-sulfonates and naphthalene-2-sulfonates.

If an inventive basic compound is isolated as a salt, the corresponding free base form of that compound may be prepared by any suitable method known to the art, including treatment of the salt with an inorganic or organic base, suitably an inorganic or organic base having a higher pKₐ than the free base form of the compound.

### General Methods of Preparation

The compounds of Formula I may be obtained by using synthetic procedures illustrated in the Schemes below or by drawing on the knowledge of a skilled organic chemist. The synthesis provided in these Schemes are applicable for producing compounds of the invention having a variety of different R¹ and R² groups employing appropriate precursors, which are suitably protected if needed, to achieve compatibility with the reactions outlined herein. Subsequent deprotection, where needed, affords compounds of the nature generally disclosed. While the Schemes are shown with compounds only of Formula I, they are illustrative of processes that may be used to make the compounds of the invention.

Compound names were generated using the software naming program ACD/Name Pro V6.02 available from Advanced Chemistry Development, Inc., 110 Yonge Street, 14^{th} Floor, Toronto, Ontario, Canada, M5C 1T4 (http://www.acdlabs.com/).

As shown in Scheme 1, the compounds of Formula I can be prepared in a multi-step sequence starting with formation of the urea moiety by reaction of a substituted phenyl isocyanate with a suitably substituted hydroxy aniline. The compounds of Formula I may be prepared by reaction of the resulting phenol-urea with 4,6-dichloropyrimidine in a suitable solvent in the presence of a base followed by treatment with methylamine.

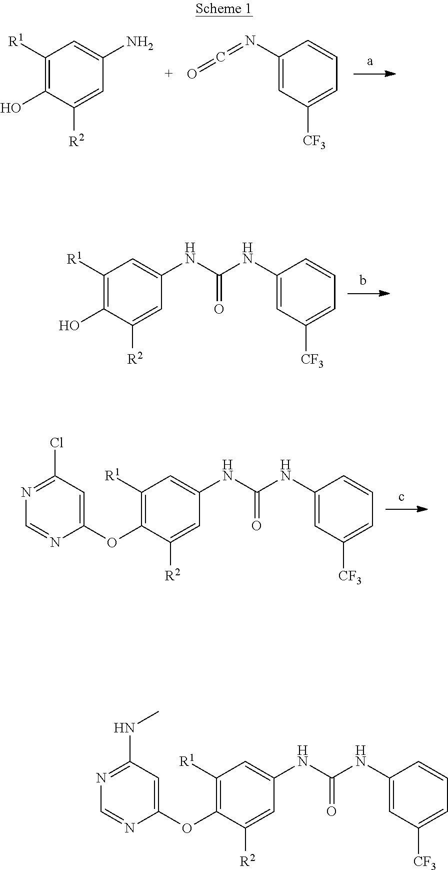

a) pyridine, rt, 2 d; b) 4,6-dichloropyrimidine, KOt-Bu, Na₂CO₃, DMF, rt, 18 h; c) CH₃NH₂ in THF, 50° C., 2.5 h or CH₃NH₂ in THF, isopropanol, μw 120° C., 20 min

Alternatively, the compounds of Formula I can be prepared via the multi-step sequence shown in Scheme 2. In this process, the pyrimidinyl-phenyl ether is prepared by reaction of a suitably substituted nitrophenol with 4,6-dichloropyrimidine. This reaction may be conducted under a variety of conditions, as noted below (a, b, c, or d). Formation of the urea is accomplished by first reducing the nitro moiety of the pyrimidinyl-phenyl ether to form the corresponding aniline, followed by reaction with a substituted phenyl isocyanate. Subsequent by treatment with methylamine provides the compounds of Formula I.

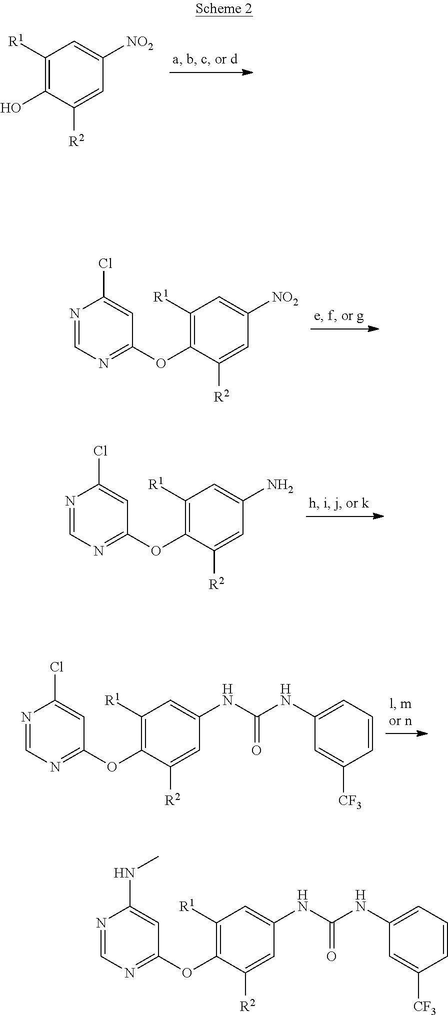

a) 4,6-dichloropyrimidine, NaOH, isopropanol, H₂O, μw, 120° C., 40 min;
b) 4,6-dichloropyrimidine, NaOH, acetone/H₂O, 45° C., 36 h; c) 4,6-dichloropyrimidine, K₂CO₃, CH₃CN, reflux, 48 h; d) 4,6-dichloropyrimidine, NaOH, DMF, μw, 140-165° C., 40 min; e) Fe powder, NH₄Cl, EtOH, reflux, 18-36 h; f) Fe powder, AcOH, reflux; g) NaBH₄, NiCl₂.6H₂O, MeOH, 0° C., 10-20 min; h) 1-isocyanato-3-(trifluoromethyl)benzene, THF, rt, overnight; i) 1-isocyanato-3-(trifluoromethyl)benzene, CH₂Cl₂, rt, 3 d; j) 1-isocyanato-3-(trifluoromethyl)benzene, dioxane, rt, 4-17 h; k) 1-isocyanato-3-(trifluoromethyl)benzene, pyridine, rt, 2 h; I) CH₃NH₂ in THF, isopropanol, μw, 100° C., 20 min; m) CH₃NH₂ in THF, 50° C., 2-3 h; n) CH₃NH₂ in EtOH, 50° C., 2 h.

The invention also includes various deuterated forms of the compounds of Formula I. Each available hydrogen atom attached to a carbon atom may be independently replaced with a deuterium atom. A person of ordinary skill in the art will know how to synthesize deuterated forms of the compounds of Formula I. For example, deuterated alkyl group amines may be prepared by conventional techniques (see for example: methyl-d₃-amine available from Aldrich Chemical Co., Milwaukee, Wis., Cat. No. 489, 689-2). Employing such compounds according to Scheme 1 or Scheme 2 will allow for the preparation of compounds of Formula I in which various hydrogen atoms are replaced with a deuterium atom.

### Methods of Use

The present invention is directed to a method of inhibiting TNNI3K which comprises contacting the kinase with a compound of Formula I or a salt thereof, particularly a pharmaceutically acceptable salt thereof. This invention is also directed to a method of treatment of a TNNI3K-mediated disease or disorder comprising administering an effective amount of the compound of Formula I or a salt thereof, particularly a pharmaceutically acceptable salt thereof, to a patient, specifically a human, in need thereof. As used herein, “patient” refers to a human or other mammal. Specifically, this invention is directed to a method of inhibiting TNNI3K activity, comprising contacting the kinase with an effective amount of a compound of Formula I or a pharmaceutically acceptable salt thereof. For example, TNNI3K activity may be inhibited in mammalian cardiac tissue by administering to a patient in need thereof, an effective amount a compound of Formula I or a pharmaceutically acceptable salt thereof.

The compounds of this invention may be particularly useful for treatment of TNNI3K-mediated diseases or disorders, specifically by inhibition of TNNI3K activity, where such diseases or disorders are selected from heart failure, particularly congestive heart failure; cardiac hypertrophy; and heart failure or congestive heart failure resulting from cardiac hypertrophy. The compounds of this invention may also be useful for the treatment of heart failure or congestive heart failure resulting from myocardial ischemia or myocardial infarction.

A therapeutically “effective amount” is intended to mean that amount of a compound that, when administered to a patient in need of such treatment, is sufficient to effect treatment, as defined herein. Thus, e.g., a therapeutically effective amount of a compound of Formula I, or a pharmaceutically acceptable salt thereof, is a quantity of an inventive agent that, when administered to a human in need thereof, is sufficient to modulate or inhibit the activity of TNNI3K such that a disease condition which is mediated by that activity is reduced, alleviated or prevented. The amount of a given compound that will correspond to such an amount will vary depending upon factors such as the particular compound (e.g., the potency (pXC₅₀), efficacy (EC₅₀), and the biological half-life of the particular compound), disease condition and its severity, the identity (e.g., age, size and weight) of the patient in need of treatment, but can nevertheless be routinely determined by one skilled in the art. Likewise, the duration of treatment and the time period of administration (time period between dosages and the timing of the dosages, e.g., before/with/after meals) of the compound will vary according to the identity of the mammal in need of treatment (e.g., weight), the particular compound and its properties (e.g., pharmaceutical characteristics), disease or condition and its severity and the specific composition and method being used, but can nevertheless be determined by one of skill in the art.

“Treating” or “treatment” is intended to mean at least the mitigation of a disease condition in a patient, where the disease condition is caused or mediated by TNNI3K. The methods of treatment for mitigation of a disease condition include the use of the compounds in this invention in any conventionally acceptable manner, for example for prevention, retardation, prophylaxis, therapy or cure of a disease. The compounds of Formula I of this invention may be useful for the treatment of heart failure, particularly congestive heart failure. The compounds of Formula I of this invention may be useful for the treatment of cardiac hypertrophy, and heart failure or congestive heart failure resulting from cardiac hypertrophy, myocardial ischemia or myocardial infarction.

The compounds of the invention may be administered by any suitable route of administration, including both systemic administration and topical administration. Systemic administration includes oral administration, parenteral administration, transdermal administration, rectal administration, and administration by inhalation. Parenteral administration refers to routes of administration other than enteral, transdermal, or by inhalation, and is typically by injection or infusion. Parenteral administration includes intravenous, intramuscular, and subcutaneous injection or infusion. Inhalation refers to administration into the patient's lungs whether inhaled through the mouth or through the nasal passages. Topical administration includes application to the skin.

The compounds of the invention may be administered once or according to a dosing regimen wherein a number of doses are administered at varying intervals of time for a given period of time. For example, doses may be administered one, two, three, or four times per day. Doses may be administered until the desired therapeutic effect is achieved or indefinitely to maintain the desired therapeutic effect. Suitable dosing regimens for a compound of the invention depend on the pharmacokinetic properties of that compound, such as absorption, distribution, and half-life, which can be determined by the skilled artisan. In addition, suitable dosing regimens, including the duration such regimens are administered, for a compound of the invention depend on the condition being treated, the severity of the condition being treated, the age and physical condition of the patient being treated, the medical history of the patient to be treated, the nature of concurrent therapy, the desired therapeutic effect, and like factors within the knowledge and expertise of the skilled artisan. It will be further understood by such skilled artisans that suitable dosing regimens may require adjustment given an individual patient's response to the dosing regimen or over time as individual patient needs change.

Treatment of TNNI3K-mediated disease conditions may be achieved using the compounds of this invention as a monotherapy, or in dual or multiple combination therapy, such as in combination with other cardiovascular agents, for example, in combination with one or more of the following agents: a beta-blocker, an ACE inhibitor, an angiotensin receptor blocker (ARB), a calcium channel blocker, a diuretic, a renin inhibitor, a centrally acting antihypertensive, a dual ACE/NEP inhibitor, an aldosterone synthase inhibitor, and an aldosterone-receptor antagonist, which are administered in effective amounts as is known in the art.

Examples of suitable beta blockers include timolol (such as BLOCARDEN™) carteolol (such as CARTROL™), carvedilol (such as COREG™), nadolol (such as CORGARD™), propanolol (such as INNOPRAN XL™), betaxolol (such as KERLONE™) penbutolol (such as LEVATOL™), metoprolol (such as LOPRESSOR™ and TOPROL-XL™), atenolol (such as TENORMIN™), pindolol (such as VISKEN™), bisoprolol, bucindolol, esmolol, acebutolol, labetalol, nebivolol, celiprolol, sotalol, and oxprenolol. Examples of suitable ACE inhibitors include alacepril, benazepril, benazaprilat, captopril, ceronapril, cilazapril, delapril, enalapril, enalaprilat, fosinopril, lisinopril, moexipiril, moveltopril, perindopril, quinapril, quinaprilat, ramipril, ramiprilat, spirapril, temocapril, trandolapril, and zofenopril. Preferred ACE inhibitors are benazepril, enalpril, lisinopril, and ramipril. Examples of suitable angiotensin receptor blockers include candesartan, eprosartan, irbesartan, losartan, olmesartan, tasosartan, telmisartan, and valsartan. Examples of suitable calcium channel blockers include dihydropyridines (DHPs) and non-DHPs. Suitable DHPs include amlodipine, felodipine, ryosidine, isradipine, lacidipine, nicardipine, nifedipine, nigulpidine, niludipine, nimodiphine, nisoldipine, nitrendipine, and nivaldipine, and their pharmaceutically acceptable salts. Suitable non-DHPs are flunarizine, prenylamine, diltiazem, fendiline, gallopamil, mibefradil, anipamil, tiapamil, and verampimil, and their pharmaceutically acceptable salts. A suitable diuretic is a thiazide derivative selected from amiloride, chlorothiazide, hydrochlorothiazide, methylchlorothiazide, and chlorothalidon. A suitable renin inhibitor is aliskiren. Examples of suitable centrally acting antiphypertensives include clonidine, guanabenz, guanfacine and methyldopa. Examples of suitable dual ACE/NEP inhibitors include omapatrilat, fasidotril, and fasidotrilat. Examples of suitable aldosterone synthase inhibitors include anastrozole, fadrozole, and exemestane. Examples of suitable aldosterone-receptor antagonists include spironolactone and eplerenone.

The invention further includes the use of compounds of the invention as an active therapeutic substance, in particular in the treatment of diseases mediated by TNNI3K. Specifically, the invention includes the use of compounds of the invention in the treatment of heart failure, particularly congestive heart failure; cardiac hypertrophy; heart failure or congestive heart failure resulting from cardiac hypertrophy; and heart failure or congestive heart failure resulting from myocardial ischemia or myocardial infarction.

In another aspect, the invention includes the use of compounds of the invention in the manufacture of a medicament for use in the treatment of the above disorders.

### Compositions

The compounds of the invention will normally, but not necessarily, be formulated into a pharmaceutical composition prior to administration to a patient. Accordingly, in another aspect the invention is directed to pharmaceutical compositions comprising a compound of the invention and a pharmaceutically acceptable excipient.

The pharmaceutical compositions of the invention may be prepared and packaged in bulk form wherein an effective amount of a compound of the invention can be extracted and then given to the patient such as with powders, syrups, and solutions for injection. Alternatively, the pharmaceutical compositions of the invention may be prepared and packaged in unit dosage form. For oral application, for example, one or more tablets or capsules may be administered. A dose of the pharmaceutical composition contains at least a therapeutically effective amount of a compound of this invention (i.e., a compound of Formula I or a salt, particularly a pharmaceutically acceptable salt, thereof). When prepared in unit dosage form, the pharmaceutical compositions may contain from 1 mg to 1000 mg of a compound of this invention.

The pharmaceutical compositions of the invention typically contain one compound of the invention. However, in certain embodiments, the pharmaceutical compositions of the invention contain more than one compound of the invention. In addition, the pharmaceutical compositions of the invention may optionally further comprise one or more additional pharmaceutically active compounds.

As used herein, “pharmaceutically acceptable excipient” means a material, composition or vehicle involved in giving form or consistency to the composition. Each excipient must be compatible with the other ingredients of the pharmaceutical composition when commingled such that interactions which would substantially reduce the efficacy of the compound of the invention when administered to a patient and interactions which would result in pharmaceutical compositions that are not pharmaceutically acceptable are avoided. In addition, each excipient must of course be of sufficiently high purity to render it pharmaceutically acceptable.

The compounds of the invention and the pharmaceutically acceptable excipient or excipients will typically be formulated into a dosage form adapted for administration to the patient by the desired route of administration. Conventional dosage forms include those adapted for (1) oral administration such as tablets, capsules, caplets, pills, troches, powders, syrups, elixirs, suspensions, solutions, emulsions, sachets, and cachets; (2) parenteral administration such as sterile solutions, suspensions, and powders for reconstitution; (3) transdermal administration such as transdermal patches; (4) rectal administration such as suppositories; (5) inhalation such as aerosols and solutions; and (6) topical administration such as creams, ointments, lotions, solutions, pastes, sprays, foams, and gels.

Suitable pharmaceutically acceptable excipients will vary depending upon the particular dosage form chosen. In addition, suitable pharmaceutically acceptable excipients may be chosen for a particular function that they may serve in the composition. For example, certain pharmaceutically acceptable excipients may be chosen for their ability to facilitate the production of uniform dosage forms. Certain pharmaceutically acceptable excipients may be chosen for their ability to facilitate the production of stable dosage forms. Certain pharmaceutically acceptable excipients may be chosen for their ability to facilitate the carrying or transporting the compound or compounds of the invention once administered to the patient from one organ, or portion of the body, to another organ, or portion of the body. Certain pharmaceutically acceptable excipients may be chosen for their ability to enhance patient compliance.

Suitable pharmaceutically acceptable excipients include the following types of excipients: diluents, fillers, binders, disintegrants, lubricants, glidants, granulating agents, coating agents, wetting agents, solvents, co-solvents, suspending agents, emulsifiers, sweeteners, flavoring agents, flavor masking agents, coloring agents, anti-caking agents, humectants, chelating agents, plasticizers, viscosity increasing agents, antioxidants, preservatives, stabilizers, surfactants, and buffering agents. The skilled artisan will appreciate that certain pharmaceutically acceptable excipients may serve more than one function and may serve alternative functions depending on how much of the excipient is present in the formulation and what other ingredients are present in the formulation.

Skilled artisans possess the knowledge and skill in the art to enable them to select suitable pharmaceutically acceptable excipients in appropriate amounts for use in the invention. In addition, there are a number of resources that are available to the skilled artisan which describe pharmaceutically acceptable excipients and may be useful in selecting suitable pharmaceutically acceptable excipients. Examples include Remington's Pharmaceutical Sciences (Mack Publishing Company), The Handbook of Pharmaceutical Additives (Gower Publishing Limited), and The Handbook of Pharmaceutical Excipients (the American Pharmaceutical Association and the Pharmaceutical Press).

The pharmaceutical compositions of the invention are prepared using techniques and methods known to those skilled in the art. Some of the methods commonly used in the art are described in Remington's Pharmaceutical Sciences (Mack Publishing Company).

In one aspect, the invention is directed to a solid oral dosage form such as a tablet or capsule comprising an effective amount of a compound of the invention and a diluent or filler. Suitable diluents and fillers include lactose, sucrose, dextrose, mannitol, sorbitol, starch (e.g. corn starch, potato starch, and pre-gelatinized starch), cellulose and its derivatives (e.g. microcrystalline cellulose), calcium sulfate, and dibasic calcium phosphate. The oral solid dosage form may further comprise a binder. Suitable binders include starch (e.g. corn starch, potato starch, and pre-gelatinized starch), gelatin, acacia, sodium alginate, alginic acid, tragacanth, guar gum, povidone, and cellulose and its derivatives (e.g. microcrystalline cellulose). The oral solid dosage form may further comprise a disintegrant. Suitable disintegrants include crospovidone, sodium starch glycolate, croscarmelose, alginic acid, and sodium carboxymethyl cellulose. The oral solid dosage form may further comprise a lubricant. Suitable lubricants include stearic acid, magnesium stearate, calcium stearate, and talc.

### EXAMPLES

The following examples illustrate the invention. These examples are not intended to limit the scope of the present invention, but rather to provide guidance to the skilled artisan to prepare and use the compounds, compositions, and methods of the present invention. While particular embodiments of the present invention are described, the skilled artisan will appreciate that various changes and modifications can be made without departing from the spirit and scope of the invention.

In the following experimental descriptions, the following abbreviations may be used:

| Abbreviation | Meaning |
|---|---|
| AcOH | acetic acid |
| Aq | aqueous |
| Brine | saturated aqueous NaCl |
| CH₂Cl₂ | methylene chloride |
| CH₃CN or MeCN | acetonitrile |
| CH₃NH₂ | methylamine |
| d | day |
| DMF | N,N-dimethylformamide |
| DMSO | dimethylsulfoxide |
| Equiv | equivalents |
| Et | ethyl |
| Et₃N | triethylamine |
| Et₂O | diethyl ether |
| EtOAc | ethyl acetate |
| EtOH | ethanol |
| ESI | electrospray ionization |
| h, hr | hour |
| HCl | hydrochloric acid |
| i-Pr₂NEt | N′,N′-diisopropylethylamine |
| KOt-Bu | potassium tert-butoxide |
| LCMS | liquid chromatography-mass spectroscopy |
| Me | methyl |
| MeOH or CH₃OH | methanol |
| MgSO₄ | magnesium sulfate |
| min | minute |
| MS | mass spectrum |
| μw | microwave |
| NaBH₄ | sodium borohydride |
| Na₂CO₃ | sodium carbonate |
| NaHCO₃ | sodium bicarbonate |
| NaOH | sodium hydroxide |
| Na₂SO₄ | sodium sulfate |
| NH₄Cl | ammonium chloride |
| NiCl₂•6H₂O | nickel (II) chloride hexahydrate |
| NMP | N-methyl-2-pyrrolidone |
| Ph | phenyl |
| rt | room temperature |
| satd | saturated |
| SCX | strong cation exchange |
| SPE | solid phase extraction |
| TFA | trifluoroacetic acid |
| THF | tetrahydrofuran |
| t_{R} | retention time |

### Preparation 1

### 2-bromo-6-(methyloxy)-4-nitrophenol

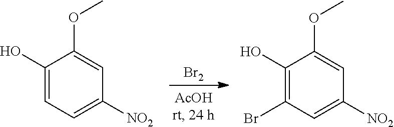

2-(Methyloxy)-4-nitrophenol (1 g, 5.91 mmol) was dissolved in glacial acetic acid (11.82 mL) with the help of sonication. Bromine (0.305 ml, 5.91 mmol) was added neat dropwise via syringe over 10 min at rt, using a septum and needle open to air. The resulting mixture was stirred overnight at room temperature. The crude reaction was pipetted slowly, carefully into well stirred water (200 mL) and the resulting mixture stirred for 10 min. The resulting solids were filtered and washed well with water (2×50 mL) and dried in a vacuum oven to afford 2-bromo-6-(methyloxy)-4-nitrophenol (1.16 g, 75% yield). ¹H NMR (400 MHz, methanol-d₄) δ 8.09 (d, J=2.76 Hz, 1H), 7.81 (d, J=2.51 Hz, 1H), 4.00 (s, 3H); MS (m/z) 248.7 (M+H⁺)

The following chloro-nitro-phenols were prepared with procedures analogous to that described in Preparation 1 using the specified nitro-phenol as a starting material and bubbling Cl₂ gas through the reaction mixture rather than adding neat Br₂:

| | Nitro-Phenol Starting | |
|---|---|---|
| Chloro-Nitro-Phenol | Material | MS (m/z) |
| 2-chloro-6-methyl- | 2-methyl-4-nitrophenol | 187.9 |
| 4-nitrophenol | | (M + H⁺) |
| 2-chloro-6-(methyloxy)- | 2-(methyloxy)-4-nitrophenol | 203.9 |
| 4-nitrophenol | | (M + H⁺) |
| 2-chloro-6-ethyl- | 2-ethyl-4-nitrophenol | 202.1 |
| 4-nitrophenol | | (M + H⁺) |
| 2-chloro-6- | 2-(trifluoromethyl)-4- | Did not ionize |
| (trifluoromethyloxy)- | nitrophenol | in ESI mode |
| 4-nitrophenol | | |

### Preparation 2

4[(6-chloro-4-pyrimidinyl)oxy]-3,5-dimethylaniline

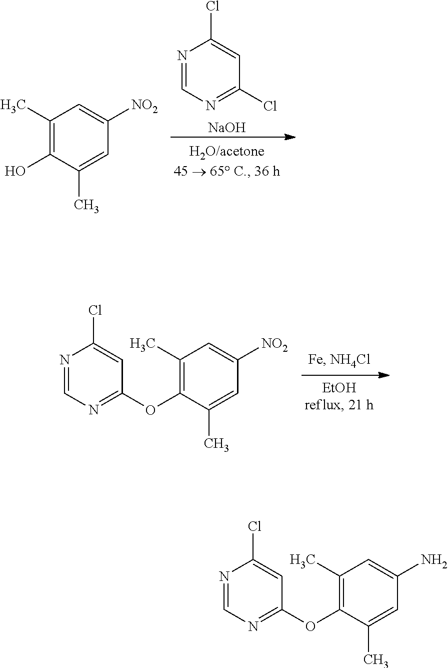

### Step 1. 4-chloro-6-[(2,6-dimethyl-4-nitrophenyl)oxy]pyrimidine

To 4,6-dichloropyrimidine (3 g, 20.14 mmol) and 2,6-dimethyl-4-aminophenol (3.37 g, 20.14 mmol) in acetone (20 mL) was added NaOH (0.805 g, 20.14 mmol) and 20 mL water. The resulting solution was then heated to 45° C. After 18 h at 45° C., starting material remained. The reaction mixture was then heated to 65° C. for 18 h. The acetone was removed in vacuo, the residue diluted with water, filtered and the filter cake washed with water. The solid was then washed with 10% sodium carbonate (3 times), then water, and dried in the vacuum oven to give 4-chloro-6-[(2,6-dimethyl-4-nitrophenyl)oxy]pyrimidine (3.81 g, 66% yield) as a brown solid. ¹H NMR (400 MHz, DMSO-d₆) δ 8.63 (s, 1H), 8.13 (s, 2H), 7.63 (s, 1H), 2.17 (s, 6H); MS (m/z) 279.9 (M+H⁺)

### Step 2. 4-[(6-chloro-4-pyrimidinyl)oxy]-3,5-dimethylaniline

To 4-chloro-6-[(2,6-dimethyl-4-nitrophenyl)oxy]pyrimidine (1.5 g, 5.36 mmol), in 80% ethanol (26.8 mL), was added iron powder (<10 micron, 1.498 g, 26.8 mmol) and ammonium chloride (1.5 g, 28.0 mmol). The resulting mixture was flushed with N₂ and heated to reflux for 21 h. The reaction mixture was then diluted with EtOH and filtered through Celite®. Most of the EtOH was then removed in vacuo and the residue treated with ethyl acetate and water. NaHCO₃ was then added and the mixture extracted with ethyl acetate. The combined organic extracts were then washed with brine, dried over Na₂SO₄, filtered and concentrated in vacuo. The crude material was purified via column chromatography (ISCO, 40 g silica column, 0-100% ethyl acetate/hexanes) to give 4-[(6-chloro-4-pyrimidinyl)oxy]-3,5-dimethylaniline (0.635 g, 61% yield) as a yellow-brown solid. MS (m/z) 249.9 (M+H⁺)

The following anilines were prepared with procedures analogous to that described in Preparation 2 using the specified phenol and 4,6-dichloropyrimidine as starting materials in Step 1 and the noted conditions for Steps 1 and 2:

| | | | | Reduction |
|---|---|---|---|---|
| | MS | Phenol used | Conditions for | Conditions used |
| Aniline | (m/z) | in Step 1 | Step 1 | in Step 2 |
| 4-[(6-chloro-4- | 239.8 | 2-fluoro-4- | NaOH, | Fe, NH₄Cl, EtOH, |
| pyrimidinyl)oxy]-3- | (M + H⁺) | nitrophenol | isopropanol, H₂O, | reflux, 36 h |
| fluoroaniline | | | μw, 140° C., 40 | |
| | | | min | |
| 3-bromo-4-[(6-chloro-4- | 331.8 | 2-bromo-6- | NaOH, DMF, μw, | Fe, NH₄Cl, EtOH, |
| pyrimidinyl)oxy]-5- | (M + H⁺) | (methyloxy)- | 140-165° C., 40 | reflux, 18 h |
| (methyloxy)aniline | | 4-nitrophenol | min | |
| 3-chloro-4-[(6-chloro-4- | 256.1 | 2-chloro-4- | K₂CO₃, CH₃CN, | NaBH₄, |
| pyrimidinyl)oxy]aniline | (M + H⁺) | nitrophenol | reflux, 20 h | NiCl₂•H₂O, |
| | | | | MeOH, 0° C., 20 |
| | | | | min |
| 3-bromo-4-[(6-chloro-4- | 301.9 | 2-bromo-4- | K₂CO₃, CH₃CN, | Fe, AcOH, reflux |
| pyrimidinyl)oxy]aniline | (M + H⁺) | nitrophenol | reflux, 48 h | |
| 4-[(6-chloro-4- | 251.9 | 2- | K₂CO₃, CH₃CN, | NaBH₄, |
| pyrimidinyl)oxy]-3- | (M + H⁺) | (methyloxy)- | reflux, 16 h | NiCl₂•H₂O, |
| (methyloxy)aniline | | 4-nitrophenol | | MeOH, 0° C., 10 |
| | | | | min |
| 3-chloro-4-[(6-chloro-4- | 270.0 | 2-chloro-6- | K₂CO₃, CH₃CN, | NaBH₄, CH₂Cl₂, |
| pyrimidinyl)oxy]-5- | (M + H⁺) | methyl-4- | 80° C., 16 h | NiCl₂•H₂O, |
| methylaniline | | nitrophenol | | MeOH, 0° C., 5 |
| | | | | min |
| 3-chloro-4-[(6-chloro-4- | 283.9 | 2-chloro-6- | K₂CO₃, CH₃CN, | NaBH₄, |
| pyrimidinyl)oxy]-5- | (M + H)⁺ | ethyl-4- | 80° C., 6 d | NiCl₂•H₂O, |
| ethylaniline | | nitrophenol | | MeOH, 0° C., 30 |
| | | | | min |

### Preparation 3

### 3-chloro-4-[(6-chloro-4-pyrimidinyl)oxy]-5-(methyloxy)aniline

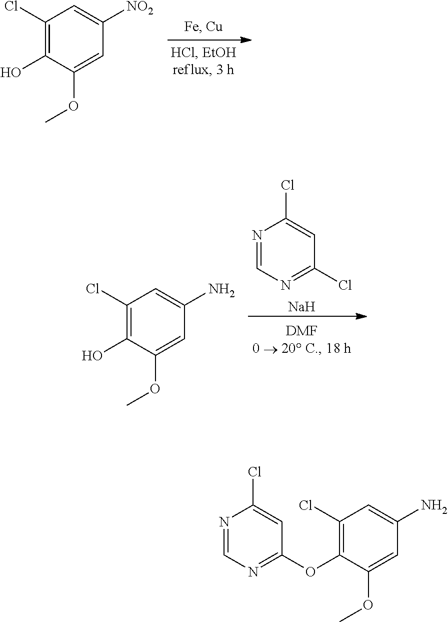

### Step 1. 4-amino-2-chloro-6-(methyloxy)phenol

To a mixture of 2-chloro-6-(methyloxy)-4-nitrophenol (8.0 g, 39.3 mmol), Fe (11.0 g, 196.5 mmol), and Cu (1.3 g, 19.6 mmol) in ethanol (200 mL), concentrated HCl (10 mL) was added. The resulting mixture was heated to reflux for 3 h and filtered. The filtrate was diluted with ethyl acetate (1 L), washed with brine (2×500 mL), dried over Na₂SO₄, filtered and concentrated in vacuo to give 4-amino-2-chloro-6-(methyloxy)phenol (5.7 g, 71% yield) as an off-white solid. MS (m/z) 174.1 (M+H⁺)

### Step 2. 3-chloro-4-[(6-chloro-4-pyrimidinyl)oxy]-5-(methyloxy)aniline

To a mixture of 4-amino-2-chloro-6-(methyloxy)phenol (4.7 g, 27.1 mmol) in DMF at 0° C., was added NaH (1.28 g, 27.1 mmol). The mixture was then stirred at 0° C. for 30 min before 4,6-dichloropyrimidine (4.03 g, 27.1 mmol) was added. The resulting mixture was then stirred at 0° C. for 30 min before warming to 20° C. and stirring for 17 h. The reaction mixture was quenched with water and combined with crude material from an additional experiment (5.76 mmol scale) run under identical conditions. The combined reaction mixtures were then diluted with ethyl acetate (1 L), washed with water (500 mL), then brine (500 mL), dried over Na₂SO₄, filtered and concentrated in vacuo to afford a brown oil. The crude material was purified via column chromatography to afford 3-chloro-4-[(6-chloro-4-pyrimidinyl)oxy]-5-(methyloxy)aniline (4.89 g total, 52% combined yield) as a brown solid. MS (m/z) 286.0 (M+H⁺)

3-Chloro-4-[(6-chloro-4-pyrimidinyl)oxy]-5-(methyloxy)aniline was prepared in a manner analogous to Preparation 3. MS (m/z) 340.0 (M+H⁺)

### Example 1

### N-(3,5-dichloro-4-{[6-(methylamino)-4-pyrimidinyl]oxy}phenyl)-N′-[3-(trifluoromethyl)phenyl]urea trifluoroacetate

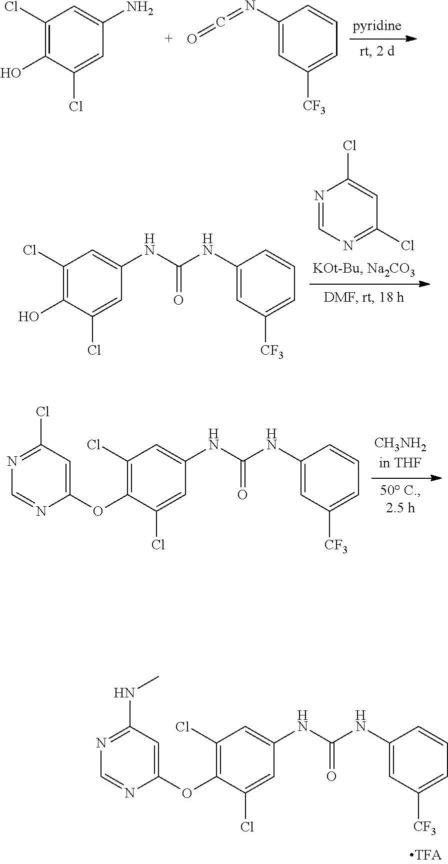

### Step 1. N-(3,5-dichloro-4-hydroxyphenyl)-N′[3-(trifluoromethyl)phenyl]urea

To a solution of 4-amino-2,6-dichlorophenol (1 g, 5.62 mmol) in anhydrous pyridine (5.62 mL) under N₂, was added neat 3-trifluoromethylphenylisocyanate (0.740 mL, 5.62 mmol) slowly dropwise. After 2 days, the reaction mixture was poured into 150 mL 1N HCl and 50 mL CH₂Cl₂. The mixture was then allowed to stand overnight at which time the product had precipitated completely. The solid was filtered and washed with CH₂Cl₂, and dried to give N-(3,5-dichloro-4-hydroxyphenyl)-N′[3-(trifluoromethyl)phenyl]urea (2.10 g, 94% yield) as a violet powder. ¹H NMR (400 MHz, DMSO-d₆) δ 9.76 (s, 1H), 9.15 (s, 1H), 8.82 (s, 1H), 8.00 (s, 1H), 7.55-7.61 (m, 1H), 7.46-7.55 (m, 3H), 7.32 (d, J=7.53 Hz, 1H)

### Step 2. N-{3,5-dichloro-4-[(6-chloro-4-pyrimidinyl)oxy]phenyl}-N′-[3-(trifluoromethyl)phenyl]urea

To a mixture of N-(3,5-dichloro-4-hydroxyphenyl)-N′[3-(trifluoromethyl)phenyl]urea (0.500 g, 1.369 mmol) and 4,6-dichloropyrimidine (0.408 g, 2.74 mmol) and N,N-dimethylformamide (DMF) (4.57 mL) was added potassium tert-butoxide (0.154 g, 1.369 mmol) and sodium carbonate (325 mesh, 0.145 g, 1.369 mmol). The mixture was then stirred at room temperature for 18 h.

The mixture was diluted with water (50 mL) and extracted with CH₂Cl₂ (3×50 mL). The combined organic extracts were then washed with water (2×50 mL) and brine (1×50 mL). The CH₂Cl₂ layer was dried over Na₂SO₄, filtered and concentrated in vacuo. The crude material was then purified via column chromatography (ISCO, 40 g silica column, 0-50% EtOAc/hexanes over 15 min) to give N-{3,5-dichloro-4-[(6-chloro-4-pyrimidinyl)oxy]phenyl}-N′-[3-(trifluoromethyl)phenyl]urea (0.440 g, 66% yield) off-white crispy foam. ¹H NMR (400 MHz, DMSO-d₆) δ 9.36 (s, 1H), 9.25 (s, 1H), 8.71 (s, 1H), 8.02 (s, 1H), 7.72-7.77 (m, 3H), 7.60-7.67 (m, 1H), 7.54 (t, J=7.91 Hz, 1H), 7.36 (d, J=7.53 Hz, 1H); MS (m/z) 366.8 (M+H⁺)

### Step 3. N-(3,5-dichloro-4-{[6-(methylamino)-4-pyrimidinyl]oxy}phenyl)-N′-[3-(trifluoromethyl)phenyl]urea trifluoroacetate

To N-{3,5-dichloro-4-[(6-chloro-4-pyrimidinyl)oxy]phenyl}-N′-[3-(trifluoromethyl)phenyl]urea (0.150 g, 0.314 mmol) was added 2M methylamine in THF (1.00 mL, 2.00 mmol). The mixture was then heated in a sealed tube to 50° C. for 2.5 h. The volatiles were then allowed to evaporate overnight by vigorous stirring while uncapped. The residue was dissolved in DMF and MeOH, filtered, and then purified via reverse phase HPLC (Waters, Sunfire, 30×100 mm column, 44-78% CH₃CN/H₂O with 0.1% TFA) to afford N-(3,5-dichloro-4-{[6-(methylamino)-4-pyrimidinyl]oxy}phenyl)-N′-[3-(trifluoromethyl)phenyl]urea trifluoroacetate (0.051 g, 26% yield).

The following compounds were prepared with procedures analogous to that described in Example 1 using 1-isocyanato-3-(trifluoromethyl)benzene and the specified amine as starting materials in Step 1:

| Ex. | Name | Structure | Aniline |
|---|---|---|---|
| 2 | N-(3,5-dibromo-4-{[6-(methylamino)-4- pyrimidinyl]oxy}phenyl)-N′-[3- (trifluoromethyl)phenyl]urea | 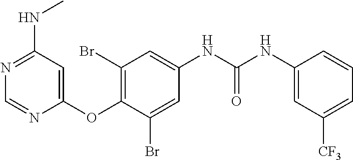 | 4-amino-2,6- dibromophenol |
| 3 | N-(3-bromo-5-chloro-4-{[6- (methylamino)-4- pyrimidinyl]oxy}phenyl)-N′-[3- (trifluoromethyl)phenyl]urea trifluoroacetate | 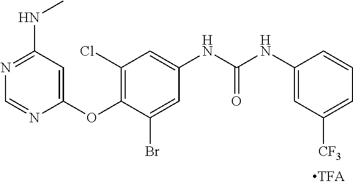 | 4-amino-2- bromo-6- chlorophenol |

### Example 4

### N-(3,5-difluoro-4-{[6-(methylamino)-4-pyrimidinyl]oxy}phenyl)-N′-[3-(trifluoromethyl)phenyl]urea

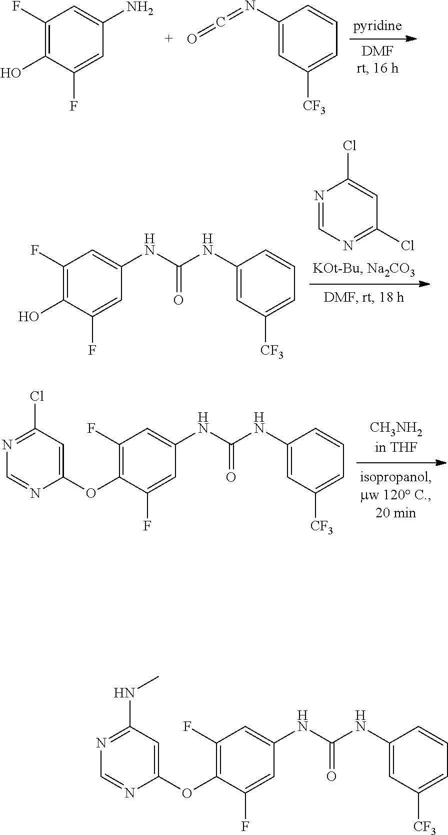

### Step 1. N-(3,5-difluoro-4-hydroxyphenyl)-N′-[3-(trifluoromethyl)phenyl]urea

To a solution of 4-amino-2,6-difluorophenol (1.6 g, 11.03 mmol) in anhydrous pyridine (8 mL) and N,N-dimethylformamide (3 mL), was added 3-trifluoromethylphenylisocyanate (1.236 mL, 8.82 mmol) slowly dropwise. The resulting solution was stirred overnight at room temperature.

The reaction mixture was pipetted into 200 mL 2N HCl, diluted with 100 mL CH₂Cl₂, filtered through a coarse frit and washed once with water. The filtrate was washed with 1N HCl and brine, dried over Na₂SO₄, filtered and concentrated in vacuo. The crude material was then purified via column chromatography (ISCO, 40 g silica column, 0-85% ethyl acetate/hexanes) to afford N-(3,5-difluoro-4-hydroxyphenyl)-N′-[3-(trifluoromethyl)phenyl]urea (0.498 g, 13% yield) as a white solid. ¹H NMR (400 MHz, DMSO-d₆) δ=9.71 (s, 1H), 9.10 (s, 1H), 8.82 (s, 1H), 7.99 (s, 1H), 7.58 (d, J=8.5 Hz, 1H), 7.51 (t, J=7.8 Hz, 1H), 7.32 (d, J=7.5 Hz, 1H), 7.23-7.11 (m, 2H); MS (m/z) 332.9 (M+H⁺)

### Step 2. N-{-4-[(6-chloro-4-pyrimidinyl)oxy]-3,5-difluorophenyl}-N′-[3-(trifluoromethyl)phenyl]urea

To a solution of N-(3,5-difluoro-4-hydroxyphenyl)-N′[3-(trifluoromethyl)phenyl]urea (0.498 g, 1.499 mmol) and 4,6-dichloropyrimidine (0.447 g, 3.00 mmol) in N,N-dimethylformamide (5.996 mL) were added potassium tert-butoxide (0.210 g, 1.874 mmol) and potassium carbonate (0.207 g, 1.499 mmol). The mixture was stirred for 1 h before an additional 1.25 equivalents of potassium tert-butoxide (0.210 g, 1.874 mmol) were added. The reaction mixture was then stirred overnight.

The mixture was diluted with ethyl acetate/ether (1:1, 50 mL) and washed with water (2×50 mL), dried over Na₂SO₄, filtered, and concentrated in vacuo. The residue was absorbed onto florisil and purified via column chromatography (ISCO, 12 g silica column, 0-50% ethyl acetate/hexanes to afford N-{-4-[(6-chloro-4-pyrimidinyl)oxy]-3,5-difluorophenyl}-N′-[3-(trifluoromethyl)phenyl]urea (0.408, 58% yield). ¹H NMR (400 MHz, DMSO-d₆) δ=9.30 (d, J=7.3 Hz, 2H), 8.72 (s, 1H), 8.01 (s, 1H), 7.74 (s, 1H), 7.62 (d, J=8.3 Hz, 1H), 7.54 (t, J=7.9 Hz, 1H), 7.42 (s, 1H), 7.44 (s, 1H), 7.36 (d, J=7.8 Hz, 1 H); MS (m/z) 445.0 (M+H⁺)

### Step 3. N-(3,5-difluoro-4-{[6-(methylamino)-4-pyrimidinyl]oxy}phenyl)-N′-[3-(trifluoromethyl)phenyl]urea

N-{4-[(6-chloro-4-pyrimidinyl)oxy]-3,5-difluorophenyl}-N′-[3-(trifluoromethyl)phenyl]urea (408 mg, 0.917 mmol) and 2M methylamine in THF (2.293 mL, 4.59 mmol) were combined in isopropanol (1 mL) and heated in the microwave at 120° C. for 20 min. The crude reaction mixture was then absorbed onto florosil and purified via column chromatography (ISCO, 12 g silica, 10-100% ethyl acetate/hexanes) to give N-(3,5-difluoro-4-{[6-(methylamino)-4-pyrimidinyl]oxy}phenyl)-N′-[3-(trifluoromethyl)phenyl]urea (0.177 g, 42%).

### Example 5

### N-(3,5-dimethyl-4-{[6-(methylamino)-4-pyrimidinyl]oxy}phenyl)-N′-[3-(trifluoromethyl)phenyl]urea trifluoroacetate

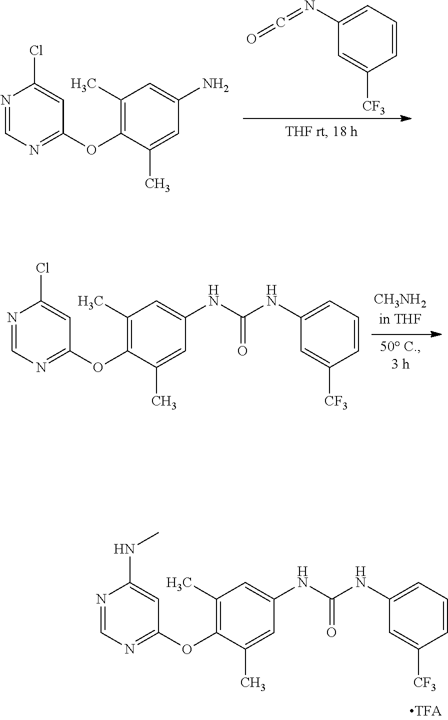

### Step 1. N-{4-[(6-chloro-4-pyrimidinyl)oxy]-3,5-dimethylphenyl}-N′-[3-(trifluoromethyl)phenyl]urea

To 4-[(6-chloro-4-pyrimidinyl)oxy]-3,5-dimethylaniline (0.635 g, 2.54 mmol) in anhydrous tetrahydrofuran (THF) (8 mL) at room temperature, was added 3-trifluoromethylphenylisocyanate (0.374 mL, 2.80 mmol) slowly dropwise. The resulting mixture was stirred overnight. In the morning, MeOH was added to dissolve the solids that had formed and to quench any excess isocyanate. The solvent was removed in vacuo and the residue purified via column chromatography (ISCO, 40 g silica column, 0-50% ethyl acetate/hexanes) to afford N-{4-[(6-chloro-4-pyrimidinyl)oxy]-3,5-dimethylphenyl}-N′[3-(trifluoromethyl)phenyl]urea (0.914 g, 77% yield) as a white solid. ¹H NMR (400 MHz, DMSO-d₆) δ 9.08 (s, 1H), 8.76 (s, 1H), 8.63 (s, 1H), 8.06 (s, 1H), 7.48-7.60 (m, 2H), 7.40 (s, 1H), 7.32 (d, J=7.03 Hz, 1H), 7.26 (s, 2H), 2.02 (s, 7H); MS (m/z) 436.9 (M+H⁺)

### Step 2. N-(3,5-dimethyl-4-{[6-(methylamino)-4-pyrimidinyl]oxy}phenyl)-N′-[3-(trifluoromethyl)phenyl]urea trifluoroacetate

N-{4-[(6-chloro-4-pyrimidinyl)oxy]-3,5-dimethylphenyl}-N′-[3-(trifluoromethyl)phenyl]urea (0.200 g, 0.458 mmol) in 2M methylamine in THF (2 mL, 4.00 mmol) was heated to 50° C. in sealed tube for 3 h. The excess methylamine and solvent was allowed to evaporate overnight. The crude material was then treated with DMF and MeOH (3 mL total volume), filtered, and purified by Waters reverse phase HPLC (Sunfire C-18 150×30 mm column, 30-70% acetonitrile/water with 0.1% TFA). Concentration of the appropriate fractions then afforded N-(3,5-dimethyl-4-{[6-(methylamino)-4-pyrimidinyl]oxy}phenyl)-N′-[3-(trifluoromethyl)phenyl]urea trifluoroacetate (0.120 g, 47%) as a white solid/colorless film.

The following compounds were prepared with procedures analogous to that described in Example 5 using 1-isocyanato-3-(trifluoromethyl)benzene and the specified aniline as starting materials in Step 1:

| Ex. | Name | Structure | Aniline |
|---|---|---|---|
| 6 | N-[3-bromo-4-{[6-(methylamino)-4- pyrimidinyl]oxy}-5-(methyloxy)phenyl]- N′-[3-(trifluoromethyl)phenyl]urea trifluoroacetate | 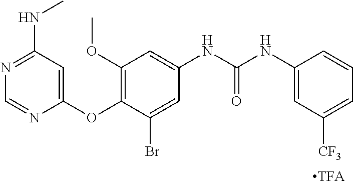 | 3-bromo-4-[(6-chloro- 4-pyrimidinyl)oxy]-5- (methyloxy)aniline |
| 7 | N-(3-chloro-4-{[6-(methylamino)-4- pyrimidinyl]oxy}phenyl)-N′-[3- (trifluoromethyl)phenyl]urea | 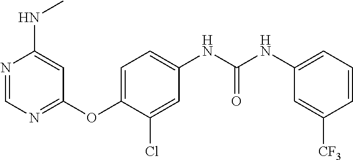 | 3-chloro-4-[(6-chloro- 4-pyrimidinyl) oxy]aniline |
| 8 | N-(3-bromo-4-{[6-(methylamino)-4- pyrimidinyl]oxy}phenyl)-N′-[3- (trifluoromethyl)phenyl]urea trifluoroacetate | 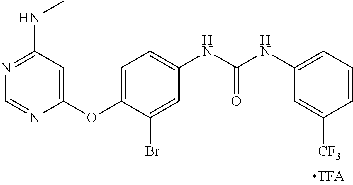 | 3-bromo-4-[(6-chloro- 4-pyrimidinyl) oxy]aniline |
| 9 | N-[4-{[6-(methylamino)-4- pyrimidinyl]oxy}-3-(methyloxy)phenyl]- N′-[3-(trifluoromethyl)phenyl]urea trifluoroacetate | 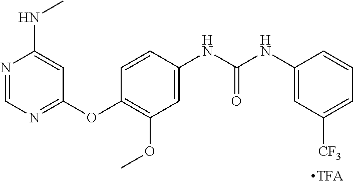 | 4-[(6-chloro-4- pyrimidinyl)oxy]-3- (methyloxy)aniline |
| 10 | N-[3-chloro-4-{[6-(methylamino)-4- pyrimidinyl]oxy}-5-(methyloxy)phenyl]- N′-[3-(trifluoromethyl)phenyl]urea trifluoroacetate | 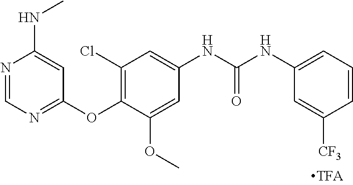 | 6-{[4-amino-2-chloro- 6-(methyloxy)phenyl] oxy}-N-methyl-4- pyrimidinamine |

### Example 11

### N-(3-fluoro-4-{[6-(methylamino)-4-pyrimidinyl]oxy}phenyl)-N′-[3-(trifluoromethyl)phenyl]urea trifluoroacetate

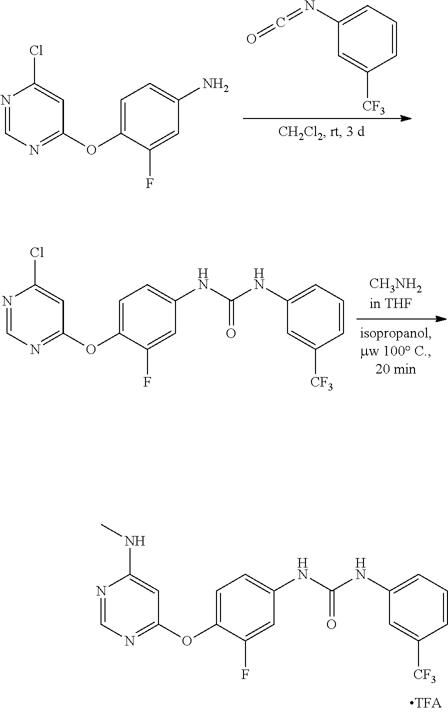

### Step 1. N-{4-[(6-chloro-4-pyrimidinyl)oxy]-3-fluorophenyl}-N′-[3-(trifluoromethyl)phenyl]urea

To a solution of 4-[(6-chloro-4-pyrimidinyl)oxy]-3-fluoroaniline (0.100 g, 0.417 mmol) in dichloromethane (4.17 mL) was added 3-trifluoromethylphenylisocyanate (0.160 g, 0.356 mmol). The resulting mixture was stirred for 3 days. The solvent was removed, and the solid suspended in 2 mL of ether, filtered, and the filter cake washed with ether (3×0.5 mL). The resulting solid was then dried under vacuum to afford N-{4-[(6-chloro-4-pyrimidinyl)oxy]-3-fluorophenyl}-N′-[3-(trifluoromethyl)phenyl]urea (0.178 g, 85% yield). ¹H NMR (400 MHz, DMSO-d₆) δ 9.17 (s, 1H), 9.11 (s, 1H), 8.68 (s, 1H), 8.02 (s, 1H), 7.68 (dd, J=2.27, 13.09 Hz, 1H), 7.50-7.64 (m, 3H), 7.30-7.37 (m, 2H), 7.20-7.27 (m, 1H); MS (m/z) 426.8 (M+H⁺)

### Step 2. N-(3-fluoro-4-{[6-(methylamino)-4-pyrimidinyl]oxy}phenyl)-N′-[3-(trifluoromethyl)phenyl]urea trifluoroacetate

A mixture of N-{4-[(6-chloro-4-pyrimidinyl)oxy]-3-fluorophenyl}-N′-[3-(trifluoromethyl)phenyl]urea (0.145 g, 0.340 mmol) and CH₃NH₂ in THF (1.699 mL, 3.40 mmol) in isopropanol (0.680 mL) was heated in the microwave at 100° C. for 20 min. The mixture was then concentrated to half of the volume to remove the methylamine, filtered and purified via HPLC (Agilent, 30×150 mm Sunfire C18 column, 25-55% CH₃CN/H₂O with 0.1% TFA,) to give N-(3-fluoro-4-{[6-(methylamino)-4-pyrimidinyl]oxy}phenyl)-N′-[3-(trifluoromethyl)phenyl]urea trifluoroacetate.

The following compounds were prepared with procedures analogous to that described in Example 11 using 1-isocyanato-3-(trifluoromethyl)benzene and the specified aniline as starting materials in Step 1. For Example 14, Step 1 was prepared using pyridine as solvent instead of CH₂Cl₂. For both Example 13 and 14, Step 2 was performed with CH₃NH₂ in ethanol at 50° C. for 2 h.

| Ex. | Name | Structure | Aniline |
|---|---|---|---|
| 12 | N-(3-chloro-5-methyl-4-{[6- (methylamino)-4- pyrimidinyl]oxy}phenyl)-N′-[3- (trifluoromethyl)phenyl]urea trifluoroacetate | 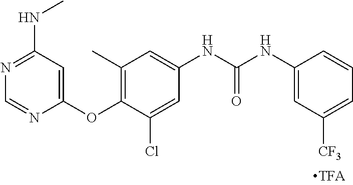 | 6-[(4-amino-2-chloro- 6-methylphenyl)oxy]- N-methyl-4- pyrimidinamine |
| 13 | N-(3-chloro-5-ethyl-4-{[6- (methylamino)-4- pyrimidinyl]oxy}phenyl)-N′-[3- (trifluoromethyl)phenyl]urea trifluoroacetate | 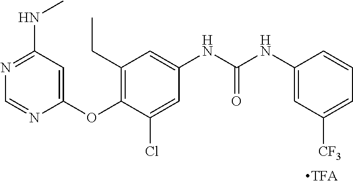 | 3-chloro-4-[(6-chloro- 4-pyrimidinyl)oxy]-5- ethylaniline |
| 14 | N-(3-chloro-4-{[6-(methylamino)- 4-pyrimidinyl]oxy}-5- (trifluoromethyloxy)phenyl)-N′-[3- (trifluoromethyl)phenyl]urea trifluoroacetate | 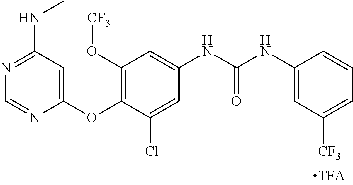 | 3-chloro-4-[(6-chloro- 4-pyrimidinyl)oxy]-5- (trifluoromethoxy) aniline |

Spectroscopic data for Examples 1-14:

| | | t_{R} | MS | |
|---|---|---|---|---|
| Ex. | Name | (min) | (m/z) | ¹H NMR |
| 1 | N-(3,5-dichloro-4-{[6- | 2.20^{a} | 472.1 | ¹H NMR (500 MHz, DMSO-d₆) δ = 9.29 |
| | (methylamino)-4- | | (M + H⁺) | (s, 1 H), 9.15 (s, 1 H), 8.08 (br. s., 1 |
| | pyrimidinyl]oxy}phenyl)-N′-[3- | | | H), 8.00 (s, 1 H), 7.67 (s, 2 H), 7.62 (d, |
| | (trifluoromethyl)phenyl]urea | | | J = 8.1 Hz, 1 H), 7.53 (t, J = 7.9 Hz, 1 |
| | trifluoroacetate | | | H), 7.45-7.38 (m, 1 H), 7.34 (d, J = |
| | | | | 7.8 Hz, 1 H), 5.98 (br. s., 1 H), 2.80 |
| | | | | (br. s., 3 H) |
| 2 | N-(3,5-dibromo-4-{[6- | 7.64^{b} | 561.7 | ¹H NMR (400 MHz, DMSO-d₆) δ 9.30 |
| | (methylamino)-4- | | (M + H⁺) | (s, 1H), 9.14 (s, 1H), 8.10 (br. s., 1H), |
| | pyrimidinyl]oxy}phenyl)-N′-[3- | | | 8.02 (s, 1H), 7.84 (s, 2H), 7.62 (d, J = |
| | (trifluoromethyl)phenyl]urea | | | 8.28 Hz, 1H), 7.54 (t, J = 8.03 Hz, 1H), |
| | | | | 7.42 (d, J = 4.77 Hz, 1H), 7.35 (d, J = |
| | | | | 7.78 Hz, 1H), 5.94 (br. s., 1H), 2.80 |
| | | | | (br. s., 3H) |
| 3 | N-(3-bromo-5-chloro-4-{[6- | 2.85^{a} | 517.8 | ¹H NMR (400 MHz, DMSO-d₆) δ = 9.35 |
| | (methylamino)-4- | | (M + H⁺) | (br. s., 1 H), 9.21 (br. s., 1 H), 8.11 (br. |
| | pyrimidinyl]oxy}phenyl)-N′-[3- | | | s., 1 H), 8.02 (s, 1 H), 7.82 (d, J = 2.3 |
| | (trifluoromethyl)phenyl]urea | | | Hz, 1 H), 7.71 (d, J = 2.3 Hz, 1 H), 7.66- |
| | trifluoroacetate | | | 7.59 (m, 1 H), 7.54 (t, J = 7.9 Hz, 1 |
| | | | | H), 7.47 (br. s., 1 H), 7.35 (d, J = 7.8 |
| | | | | Hz, 1 H), 5.97 (br. s., 1 H), 2.81 (br. s., |
| | | | | 3H) |
| 4 | N-(3,5-difluoro-4-{[6- | 2.73^{a} | 440.1 | ¹H NMR (400 MHz, DMSO-d₆) δ 9.28 |
| | (methylamino)-4- | | (M + H⁺) | (s, 1H), 9.22 (s, 1H), 8.10 (br. s., 1H), |
| | pyrimidinyl]oxy}phenyl)-N′-[3- | | | 8.00 (s, 1H), 7.61 (d, J = 8.56 Hz, 1H), |
| | (trifluoromethyl)phenyl]urea | | | 7.54 (t, J = 7.93 Hz, 1H), 7.46 (q, J = |
| | | | | 3.94 Hz, 1H), 7.30-7.40 (m, 3H), 6.03 |
| | | | | (s, 1H), 2.80 (br. s., 3H) |
| 5 | N-(3,5-dimethyl-4-{[6- | 6.77^{b} | 432.0 | ¹H NMR (400 MHz, methanol-d₄) δ |
| | (methylamino)-4- | | (M + H⁺) | 8.40 (br. s., 1H), 7.96 (s, 1H), 7.61 (d, |
| | pyrimidinyl]oxy}phenyl)-N′-[3- | | | J = 8.28 Hz, 1H), 7.48 (t, J = 7.91 Hz, |
| | (trifluoromethyl)phenyl]urea | | | 1H), 7.31 (s, 3H), 5.70 (br. s., 1H), |
| | trifluoroacetate | | | 2.97 (br. s., 3H), 2.16 (s, 6H) |
| 6 | N-[3-bromo-4-{[6- | 2.00^{a} | 514.1 | ¹H NMR (400 MHz, DMSO-d₆) δ 9.19 |
| | (methylamino)-4- | | (M + H⁺) | (s, 1H), 9.03 (br. s., 1H), 8.08 (br. s., |
| | pyrimidinyl]oxy}-5- | | | 1H), 8.03 (s, 1H), 7.58-7.65 (m, 1H), |
| | (methyloxy)phenyl]-N′-[3- | | | 7.53 (t, J = 7.78 Hz, 1H), 7.42 (d, J = |
| | (trifluoromethyl)phenyl]urea | | | 2.26 Hz, 1H), 7.34 (d, J = 7.53 Hz, 2H), |
| | trifluoroacetate | | | 7.22-7.29 (m, 1H), 5.83 (br. s., 1H), |
| | | | | 3.73 (s, 3H), 2.79 (br. s., 3H) |
| 7 | N-(3-chloro-4-{[6- | 1.27^{c} | 440.0 | ¹H NMR (400 MHz, DMSO-d₆) δ 9.79 |
| | (methylamino)-4- | | (M + H⁺) | (s, 1H), 9.69 (br. s., 1H), 8.09 (br. s., |
| | pyrimidinyl]oxy}phenyl)-N′-[3- | | | 1H), 8.00 (s, 1H), 7.80 (d, J = 2.26 Hz, |
| | (trifluoromethyl)phenyl]urea | | | 1H), 7.59 (d, J = 7.77 Hz, 1H), 7.50 (t, |
| | | | | J = 8.10 Hz, 1H), 7.34 (dd, J = 2.45, |
| | | | | 8.68 Hz, 2H), 7.29 (d, J = 7.83 Hz, 1H), |
| | | | | 7.21 (d, J = 8.76 Hz, 1H), 5.80 (br. s., |
| | | | | 1H), 2.76 (br. s., 3H) |
| 8 | N-(3-bromo-4-{[6- | 1.28^{c} | 484.0 | ¹H NMR (400 MHz, DMSO-d₆) δ 9.24 |
| | (methylamino)-4- | | (M + H⁺) | (s, 1H), 9.12 (br. s., 1H), 8.12 (br. s., |
| | pyrimidinyl]oxy}phenyl)-N′-[3- | | | 1H), 8.01 (br. s., 1H), 7.96 (br. s., 1H), |
| | (trifluoromethyl)phenyl]urea | | | 7.59 (d, J = 8.03 Hz, 1H), 7.51 (t, J = |
| | trifluoroacetate | | | 7.91 Hz, 1H), 7.35-7.44 (m, 2H), 7.32 |
| | | | | (d, J = 7.03 Hz, 1H), 7.20 (d, J = 8.53 |
| | | | | Hz, 1H), 5.81 (br. s., 1H), 2.78 (br. s., |
| | | | | 3H) |
| 9 | N-[4-{[6-(methylamino)-4- | 1.15^{c} | 434.2 | ¹H NMR (400 MHz, DMSO-d₆) δ 9.23 |
| | pyrimidinyl]oxy}-3- | | (M + H⁺) | (s, 1H), 9.06 (br. s., 1H), 8.22 (br. s., |
| | (methyloxy)phenyl]-N′-[3- | | | 1H), 8.03 (s, 1H), 7.66 (br. s., 1H), |
| | (trifluoromethyl)phenyl]urea | | | 7.58 (d, J = 8.03 Hz, 1H), 7.51 (t, J = |
| | trifluoroacetate | | | 7.91 Hz, 1H), 7.45 (s, 1H), 7.31 (d, J = |
| | | | | 7.53 Hz, 1H), 7.06 (d, J = 8.53 Hz, 1H), |
| | | | | 6.92-7.00 (m, 1H), 5.74 (br. s., 1H), |
| | | | | 3.71 (s, 3H), 2.78 (br. s., 3H) |
| 10 | N-[3-chloro-4-{[6- | 1.27^{c} | 468.0 | ¹H NMR (400 MHz, DMSO-d₆) δ 9.24 |
| | (methylamino)-4- | | (M + H⁺) | (s, 1H), 9.10 (s, 1H), 8.05 (br. s., 1H), |
| | pyrimidinyl]oxy}-5- | | | 7.99 (s, 1H), 7.58 (s, 1H), 7.49 (t, J = |
| | (methyloxy)phenyl]-N′-[3- | | | 7.99 Hz, 1H), 7.35 (br. s., 1H), 7.29 (d, |
| | (trifluoromethyl)phenyl]urea | | | J = 7.72 Hz, 1H), 7.24 (d, J = 2.32 Hz, |
| | trifluoroacetate | | | 1H), 7.19 (d, J = 2.21 Hz, 1H), 5.82 (br. |
| | | | | s., 1H), 3.69 (s, 3H), 2.75 (br. s., 3H) |
| 11 | N-(3-fluoro-4-{[6-(methylamino)- | 2.23^{a} | 421.9 | ¹H NMR (400 MHz, methanol-d₄) δ |
| | 4-pyrimidinyl]oxy}phenyl)-N′-[3- | | (M + H⁺) | 8.29 (br. s., 1H), 7.93 (br. s., 1H), 7.60- |
| | (trifluoromethyl)phenyl]urea | | | 7.72 (m, 2H), 7.50 (s, 1H), 7.33 (d, J = |
| | trifluoroacetate | | | 7.55 Hz, 1H), 7.19-7.30 (m, 2H), |
| | | | | 5.96 (br. s., 1H), 2.96 (br. s., 3H) |
| 12 | N-(3-chloro-5-methyl-4-{[6- | 2.65^{a} | 452.1 | ¹H NMR (400 MHz, DMSO-d₆) δ 9.21 |
| | (methylamino)-4- | | (M + H⁺) | (s, 1H), 9.00 (s, 1H), 8.11 (br. s., 1H), |
| | pyrimidinyl]oxy}phenyl)-N′-[3- | | | 8.03 (s, 1H), 7.57-7.66 (m, 2H), 7.49- |
| | (trifluoromethyl)phenyl]urea | | | 7.57 (m, 1H), 7.42 (br. s., 1H), 7.33 (d, |
| | trifluoroacetate | | | J = 7.55 Hz, 1H), 7.25-7.30 (m, 1H), |
| | | | | 5.86 (br. s., 1H), 2.79 (br. s., 3H), 2.09 |
| | | | | (s, 3H) |
| 13 | N-(3-chloro-5-ethyl-4-{[6- | 1.33^{c} | 466.1 | ¹H NMR (400 MHz, DMSO-d₆) δ 9.39 |
| | (methylamino)-4- | | (M + H⁺) | (s, 1H), 9.21 (s, 1H), 8.1 (br. s., 1H), |
| | pyrimidinyl]oxy}phenyl)-N′-[3- | | | 8.01 (s, 1H), 7.61 (s, 1H), 7.57-7.60 |
| | (trifluoromethyl)phenyl]urea | | | (m, 1 H), 7.51 (t, J = 7.60 Hz, 1 H), |
| | trifluoroacetate | | | 7.36 (br. s., 1 H), 7.27-7.31 (m, 1 H), |
| | | | | 5.90 (br. s., 1 H), 2.77 (br. s., 3 H), |
| | | | | 2.42 (q, J = 7.60 Hz, 2 H), 1.07 (t, J = |
| | | | | 7.60 Hz, 3 H) |
| 14 | N-(3-chloro-4-{[6- | 1.41 | 522.1 | ¹H NMR (400 MHz, DMSO-d₆) δ 9.37 |
| | (methylamino)-4- | | (M + H⁺) | (br. s., 2H), 8.04 (br. s., 1H), 7.98 (br. |
| | pyrimidinyl]oxy}-5- | | | s., 1H), 7.60-7.69 (m, 2H), 7.45-7.56 |
| | (trifluoromethyloxy)phenyl)-N′- | | | (m, 3H), 7.33 (d, J = 7.20 Hz, 1H), 5.98 |
| | [3-(trifluoromethyl)phenyl]urea | | | (br. s., 1 H), 2.78 (br. s., 3H) |
| | trifluoroacetate | | | |

^{a}LCMS Method: Agilent 1100 Series LC/MSD SL or VL using electrospray positive [ES + ve to give M + H⁺] equipped with a Sunfire C18 5.0 μm column (3.0 mm × 50 mm, i.d.), eluting with 0.05% TFA in water (solvent A) and 0.05% TFA in acetonitrile (solvent B), using the following elution gradient: 10%-100% (solvent B) over 2.5 minutes and holding at 100% for 1.7 minutes at a flow rate of 1.0 mL/minutes.
^{b}LCMS Method: Agilent 1100 Series LC/MSD SL or VL using electrospray positive [ES + ve to give M + H⁺] equipped with a Sunfire C18 5.0 μm column (3.0 mm × 50 mm, i.d.), eluting with 0.05% TFA in water (solvent A) and 0.05% TFA in acetonitrile (solvent B), using the following elution gradient 10%-100% (solvent B) over 10.0 minutes and holding at 100% for 1.7 minutes at a flow rate of 1.0 mL/minutes.
^{c}LCMS Method: On an Agilent 1200 Series LC/MSD VL using electrospray positive [ES + ve to give M + H⁺] equipped with a shim-pack XR-ODS 2.2 μm column (3.0 mm × 30 mm, 3.0 mm i.d.) eluting with 0.0375% TFA in water (solvent A) and 0.01875% TFA in acetonitrile (solvent B), using the following elution gradient 10-80% (solvent B) over 0.9 minutes and holding at 80% for 0.6 minutes at a flow rate of 1.2 mL/minutes.

### Pharmaceutical Compositions

### Example A

Tablets are prepared using conventional methods and are formulated as follows:

| Ingredient | Amount per tablet |
|---|---|
| Compound of Example I | 5 mg |
| Microcrystalline cellulose | 100 mg |
| Lactose | 100 mg |
| Sodium starch glycollate | 30 mg |
| Magnesium stearate | 2 mg |
| Total | 237 mg |

### Example B

Capsules are prepared using conventional methods and are formulated as follows:

| Ingredient | Amount per tablet |
|---|---|
| Compound of Example 3 | 15 mg |
| Dried starch | 178 mg |
| Magnesium stearate | 2 mg |
| Total | 195 mg |

### Biological Assay(s)

Materials: His-MBP-TEV-Full length human TNNI3K (hTNNI3K) was expressed in Baculokinase system and purified from amylase affinity column followed by Superdex200. The fluorescent ligand 5-({[2-({[3-({4-[(5-hydroxy-2-methylphenyl)amino]-2-pyrimidinyl}amino)phenyl]carbonyl}amino)ethyl]amino}carbonyl)-2-(6-hydroxy-3-oxo-3H-xanthen-9-yl)benzoic acid was used. The other buffer components, including MgCl₂ (Catalog Number M1028), Bis-Tris (Catalog Number B7535), DTT (Catalog Number D9779) and Chaps (Catalog Number C3023) were purchased from Sigma-Aldrich.

### Preparation of 5-({[2-({[3-({4-[(5-hydroxy-2-methylphenyl)amino]-2-pyrimidinyl}amino)phenyl]carbonyl}amino)ethyl]amino}carbonyl)-2-(6-hydroxy-3-oxo-3H-xanthen-9-yl)benzoic acid

### Step 1. 1-methyl-2-nitro-4-(2-propen-1-yloxy)benzene

A mixture of 4-methyl-3-nitrophenol (20 g, 131 mmol), potassium carbonate (27.1 g, 196 mmol), sodium iodide (21.53 g, 144 mmol), and allyl bromide (12.43 mL, 144 mmol) in acetone (278 mL) was heated to 55° C. for 3 d. The reaction mixture was cooled to room temperature, filtered and concentrated in vacuo. The residue was dissolved in 200 mL ether and washed with saturated aqueous sodium bicarbonate (2×100 mL) then brine (100 mL), dried over MgSO₄, filtered, and concentrated to afford 1-methyl-2-nitro-4-(2-propen-1-yloxy)benzene (25.8 g, 97% yield) as an orange oil. ¹H NMR (400 MHz, CDCl₃) δ 7.54 (d, J=2.77 Hz, 1H), 7.25 (d, J=8.56 Hz, 1H), 7.10 (dd, J=2.77, 8.56 Hz, 1H), 6.00-6.11 (m, J=5.26, 5.26, 10.48, 17.28 Hz, 1H), 5.42-5.49 (m, 1H), 5.31-5.38 (m, 1H), 4.60 (dt, J=1.51, 5.29 Hz, 2H), 2.55 (s, 3H); MS (m/z) 194.2 (M+H⁺)

### Step 2. 2-methyl-5-(2-propen-1-yloxy)aniline

To a mixture of 1-methyl-2-nitro-4-(2-propen-1-yloxy)benzene (25.2 g, 130 mmol) in ethanol (280 mL), water (28 mL), and acetic acid (5.6 mL, 98 mmol), was added iron (29.1 g, 522 mmol) in six portions. The resulting mixture was stirred at room temperature for 3 d. Additional acetic acid (5.6 ml, 98 mmol) and iron (7.28 g) were then added. After 6 h, an additional 3 equiv. of iron (21.82 g) was added and the mixture stirred for 17 h. The mixture was filtered through Celite®, washed with ethanol, then water, and concentrated in vacuo to remove the ethanol. The remaining aqueous mixture was then partitioned between ether (300 mL) and 2N HCl (100 mL). The layers were separated and the ethereal layer extracted with 2N HCl (2×100 mL). The combined aqueous layers were treated with NaOH until the solution reached pH 9. This mixture was diluted with methylene chloride (300 mL) and filtered. The layers were then separated. The aqueous layer was then extracted with methylene chloride (2×100 mL). The combined organic extracts were dried over MgSO₄, filtered, and concentrated in vacuo. The crude material was purified via column chromatography (ISCO, 120 g silica column, 5-30% ethyl acetate/hexanes) to afford 2-methyl-5-(2-propen-1-yloxy)aniline (11.8 g, 50% yield) as a dark red oil. ¹H NMR (400 MHz, CDCl₃) δ 7.28 (s, 1H), 7.02 (d, J=8.31 Hz, 1H), 6.59 (d, J=2.52 Hz, 1H), 6.49 (dd, J=2.52, 8.31 Hz, 1H), 5.98-6.13 (m, 1H), 5.42 (dq, J=1.53, 17.31 Hz, 1H), 5.29 (dq, J=1.38, 10.45 Hz, 1H), 4.51 (dt, J=1.51, 5.29 Hz, 2H), 2.24 (s, 3H); MS (m/z) 164.1 (M+H⁺)

### Step 3. 2-chloro-N-[2-methyl-5-(2-propen-1-yloxy)phenyl]-4-pyrimidinamine

To a solution of 2-methyl-5-(2-propen-1-yloxy)aniline (11.8 g, 72.3 mmol) in tert-butanol (103 mL), was added 2,4-dichloropyrimidine (10.77 g, 72.3 mmol) followed by sodium bicarbonate (18.22 g, 217 mmol). The resulting mixture was heated to 80° C. for 17 h. Additional 2,4-dichloropyrimidine (0.25 eq., 2.69 g) was then added and the reaction mixture stirred for 6 h. Additional 2,4-dichloropyrimidine (0.25 eq., 2.69 g) was added and the mixture stirred for 6 d. A third portion of 2,4-dichloropyrimidine (0.25 eq., 2.69 g) was then added and the mixture stirred for another 3 days. The mixture was cooled to room temperature and diluted with ethyl acetate (200 mL) and water (200 mL). The layers were separated and the aqueous layer extracted with ethyl acetate (2×100 mL). The combined organic extracts were washed with brine (100 mL), dried over Na₂SO₄, filtered, and concentrated. The crude material was purified via column chromatography (ISCO, 330 g silica column, 1-20% ethyl acetate/hexanes) to afford 2-chloro-N-[2-methyl-5-(2-propen-1-yloxy)phenyl]-4-pyrimidinamine (15.1 g, 72% yield). ¹H NMR (400 MHz, CDCl₃) δ 8.10 (d, J=5.79 Hz, 1H), 7.21 (d, J=8.56 Hz, 1H), 7.14 (br. s., 1H), 6.89 (d, J=2.52 Hz, 1H), 6.83 (dd, J=2.64, 8.44 Hz, 1H), 6.36 (d, J=5.79 Hz, 1H), 5.99-6.11 (m, 1H), 5.42 (dd, J=1.51, 17.37 Hz, 1H), 5.32 (dd, J=1.26, 10.32 Hz, 1H), 4.54 (d, J=5.29 Hz, 2H), 2.20 (s, 3H); MS (m/z) 276.1 (M+H⁺)

### Step 4. 3-[(4-{[2-methyl-5-(2-propen-1-yloxy)phenyl]amino}-2-pyrimidinyl)amino]benzoic acid

A mixture of 2-chloro-N-[2-methyl-5-(2-propen-1-yloxy)phenyl]-4-pyrimidinamine (8 g, 29.0 mmol), 3-aminobenzoic acid (3.98 g, 29.0 mmol), and HCl (14.51 mL, 29.0 mmol) in acetone (58.0 mL) and water (58.0 mL) was heated to 60° C. for 2 d. The mixture was cooled to room temperature. A stream of air was passed over the solution for one minute and a thick solid formed. The mixture was diluted with water (150 mL), filtered, washed with water (3×50 mL) and dried under vacuum to give 3-[(4-{[2-methyl-5-(2-propen-1-yloxy)phenyl]amino}-2-pyrimidinyl)amino]benzoic acid (7.4 g, 10.92 g). ¹H NMR (400 MHz, methanol-d₄) δ 8.02 (br. s., 1H), 7.79-7.88 (m, 2H), 7.77 (d, J=8.03 Hz, 1H), 7.38 (t, J=7.40 Hz, 1H), 7.24 (d, J=8.28 Hz, 1H), 6.96 (d, J=2.51 Hz, 1H), 6.89 (dd, J=2.51, 8.53 Hz, 1H), 6.35 (br. s., 1H), 5.97-6.09 (m, 1H), 5.37 (dd, J=1.63, 17.19 Hz, 1H), 5.24 (dd, J=1.25, 10.54 Hz, 1H), 4.48 (d, J=5.27 Hz, 2H), 2.21 (s, 3H); MS (m/z) 377.1 (M+H⁺)

### Step 5. 1,1-dimethylethyl{2-[({3-[(4-{[2-methyl-5-(2-propen-1-yloxy)phenyl]amino}-2-pyrimidinyl)amino]phenyl}carbonyl)amino]ethyl}carbamate

To a mixture of 3-[(4-{[2-methyl-5-(2-propen-1-yloxy)phenyl]amino}-2-pyrimidinyl)amino]benzoic acid (6.83 g, 18.15 mmol) in N,N-dimethylformamide (51.8 mL), was added N,N-diisopropylethylamine (9.51 mL, 54.4 mmol) followed by N-(2-aminoethyl) carbamic acid tert-butyl ester (3.20 g, 19.96 mmol) and HATU (8.28 g, 21.77 mmol). The resulting mixture was then stirred at room temperature for 18 h. The mixture was then diluted with ethyl acetate/ether (400 mL, 1:1), washed with water (3×300 mL), then brine, dried over Na₂SO₄, filtered, and concentrated in vacuo to give 1,1-dimethylethyl{2-[({3-[(4-{[2-methyl-5-(2-propen-1-yloxy)phenyl]amino}-2-pyrimidinyl)amino]phenyl}carbonyl)amino]ethyl}carbamate (8.3 g, 84% yield). ¹H NMR (400 MHz, DMSO-d₆) δ 9.17 (s, 1H), 8.66 (s, 1H), 8.26 (t, J=5.40 Hz, 1H), 7.93-8.04 (m, 3H), 7.28 (d, J=7.78 Hz, 1H), 7.12-7.22 (m, 2H), 7.09 (d, J=2.51 Hz, 1H), 6.90 (t, J=5.65 Hz, 1H), 6.73 (dd, J=2.64, 8.41 Hz, 1H), 6.13 (d, J=5.77 Hz, 1H), 5.94-6.08 (m, 1H), 5.37 (dd, J=1.76, 17.32 Hz, 1H), 5.24 (dd, J=1.51, 10.54 Hz, 1H), 4.51 (d, J=5.27 Hz, 2H), 3.27 (q, J=6.19 Hz, 2H), 3.09 (q, J=6.19 Hz, 2H), 2.15 (s, 3H), 1.38 (s, 9H); MS (m/z) 519.2 (M+H⁺)

### Step 6. 1,1-dimethylethyl[2-({[3-({4-[(5-hydroxy-2-methylphenyl)amino]-2-pyrimidinyl}amino)phenyl]carbonyl}amino)ethyl]carbamate

A solution of 1,1-dimethylethyl{2-[({3-[(4-{[2-methyl-5-(2-propen-1-yloxy)phenyl]amino}-2-pyrimidinyl)amino]phenyl}carbonyl)amino]ethyl}carbamate (5.5 g, 10.61 mmol) and morpholine (1.016 ml, 11.67 mmol) in N,N-dimethylformamide (42.4 mL) was degassed with nitrogen. Then tetrakis(triphenylphosphine)palladium(0) (1.226 g, 1.061 mmol) was added. The resulting mixture was heated to 80° C. for 3 h. The mixture was cooled to room temperature, diluted with ethyl acetate (250 mL), washed with water (3×200 mL), then brine (100 mL), dried over Na₂SO₄, filtered, and concentrated in vacuo until about 50 mL of ethyl acetate remained. The mixture was then allowed to stand overnight, during which time the product precipitated out. The mixture was diluted with ether (50 mL), filtered, and then washed with ether to afford 1,1-dimethylethyl[2-({[3-({4-[(5-hydroxy-2-methylphenyl)amino]-2-pyrimidinyl}amino)phenyl]carbonyl}amino) ethyl]carbamate (4.75 g, 84% yield) as a orange sticky solid. An additional 0.359 g (7% yield) was isolated from the side of the funnel. ¹H NMR (400 MHz, methanol-d₄) δ 8.11 (s, 1H), 7.91 (d, J=6.04 Hz, 1H), 7.75 (d, J=7.30 Hz, 1H), 7.35-7.41 (m, 1H), 7.30 (t, J=7.81 Hz, 1H), 7.10 (d, J=8.31 Hz, 1H), 6.87 (d, J=2.52 Hz, 1H), 6.65 (dd, J=2.64, 8.18 Hz, 1H), 6.05 (d, J=6.04 Hz, 1H), 3.46 (t, J=6.04 Hz, 2H), 3.29 (t, J=6.17 Hz, 2H), 2.17 (s, 3H), 1.42 (s, 9H); MS (m/z) 479.2 (M+H⁺)

### Step 7. N-(2-aminoethyl)-3-({4-[(5-hydroxy-2-methylphenyl)amino]-2-pyrimidinyl}amino)benzamide

A solution of 1,1-dimethylethyl[2-({[3-({4-[(5-hydroxy-2-methylphenyl)amino]-2-pyrimidinyl}amino)phenyl]carbonyl}amino)ethyl]carbamate (4.75 g, 8.93 mmol) in methylene chloride (28.6 mL) and trifluoroacetic acid (TFA) (7.15 mL) was stirred at room temperature for 18 h. The solvent was then removed under reduced pressure to afford N-(2-aminoethyl)-3-({4-[(5-hydroxy-2-methylphenyl)amino]-2-pyrimidinyl}amino)benzamide (6.5 g, 96% yield) as a brown foam. ¹H NMR (400 MHz, DMSO-d₆) δ 10.69 (br. s., 1H), 10.25 (br. s., 1H), 8.60 (t, J=5.54 Hz, 1H), 7.98 (d, J=7.05 Hz, 1H), 7.86 (br. s., 4H), 7.75 (br. s., 1H), 7.56 (d, J=7.55 Hz, 1H), 7.30 (t, J=6.67 Hz, 1H), 7.10 (d, J=8.31 Hz, 1H), 6.77 (s, 1H), 6.68 (dd, J=2.39, 8.18 Hz, 1H), 6.32 (br. s., 1H), 3.50 (q, J=6.13 Hz, 2H), 2.93-3.04 (m, 2H), 2.08 (s, 3H); MS (m/z) 379.1 (M+H⁺)

### Step 8. 5-({[2-({[3-({4-[(5-hydroxy-2-methyl phenyl)amino]-2-pyrimidinyl}amino)phenyl]carbonyl}amino)ethyl]amino}carbonyl)-2-(6-hydroxy-3-oxo-3H-xanthen-9-yl)benzoic acid

A mixture of N-(2-aminoethyl)-3-({4-[(5-hydroxy-2-methylphenyl)amino]-2-pyrimidinyl}amino)benzamide (1 g, 1.319 mmol), 4-(6-hydroxy-3-oxo-3H-xanthen-9-yl)-1,3-benzenedicarboxylic acid (0.397 g, 1.055 mmol), triethylamine (0.919 mL, 6.60 mmol), EDC (0.506 g, 2.64 mmol), and HOBT (0.202 g, 1.319 mmol) in DMF (13.19 mL) was stirred overnight at room temperature. The reaction mixture was then acidified with 2N HCl to pH 3 and diluted with ethyl acetate (100 mL). The organic layer was separated, washed with water (1×50 mL), dried over Na₂SO₄, filtered and concentrated in vacuo. This material was then combined with that from a separate experiment (0.132 mmol scale) run under identical conditions and purified via reverse phase HPLC (Agilent, 30×75 mm Sunfire C18 column, 10-40% CH₃CN/H₂O with 0.1% TFA). This material was then purified a second time via reverse phase HPLC (Restek 30×100 mm PFPp (pentafluorophenyl propyl) column, 10-90% MeCN/H₂O) to afford 5-({[2-({[3-({4-[(5-hydroxy-2-methylphenyl)amino]-2-pyrimidinyl}amino)phenyl]carbonyl}amino) ethyl]amino}carbonyl)-2-(6-hydroxy-3-oxo-3H-xanthen-9-yl)benzoic acid (0.055 g, 5% yield) as a bright orange solid. ¹H NMR (400 MHz, methanol-d₄) δ 8.46 (s, 1H), 8.17 (dd, J=1.51, 8.06 Hz, 1H), 8.14 (br. s., 1H), 7.88 (d, J=6.04 Hz, 1H), 7.71-7.77 (m, 1H), 7.38-7.45 (m, 1H), 7.26-7.36 (m, 2H), 7.08 (d, J=8.31 Hz, 1H), 6.84 (d, J=2.52 Hz, 1H), 6.70 (d, J=2.27 Hz, 2H), 6.62-6.67 (m, 3H), 6.55 (dd, J=2.27, 8.81 Hz, 2H), 6.02 (d, J=6.04 Hz, 1H), 3.69 (br. s., 4H), 2.14 (s, 3H); MS (m/z) 737.2 (M+H⁺)

### Biological Assay Method I:

A fluorescent polarization assay was used to determine does response of compound inhibition on hTNNI3K ATP binding. The binding of 5-({[2-({[3-({4-[(5-hydroxy-2-methylphenyl)amino]-2-pyrimidinyl}amino)phenyl]carbonyl}amino)ethyl]amino}carbonyl)-2-(6-hydroxy-3-oxo-3H-xanthen-9-yl)benzoic acid to the hTNNI3K ATP binding pocket results in increase of fluorescent polarization and the displacement of 5-({[2-({[3-({4-[(5-hydroxy-2-methylphenyl)amino]-2-pyrimidinyl}amino)phenyl]carbonyl}amino)ethyl]amino}carbonyl)-2-(6-hydroxy-3-oxo-3H-xanthen-9-yl)benzoic acid by a competitive compound leads to fluorescent polarization decrease.

Solution 1: Ten (10) mL of a 5 nM 5-({[2-({[3-({4-[(5-hydroxy-2-methylphenyl)amino]-2-pyrimidinyl}amino)phenyl]carbonyl}amino)ethyl]amino}carbonyl)-2-(6-hydroxy-3-oxo-3H-xanthen-9-yl)benzoic acid solution (Solution 1) was prepared by mixing 5 μL of 1M DTT and 80 μL of 10% (w/v) Chaps and 5 μL of a 10 μM 5-({[2-({[3-({4-[(5-hydroxy-2-methylphenyl)amino]-2-pyrimidinyl}amino)phenyl]carbonyl}amino) ethyl]amino}carbonyl)-2-(6-hydroxy-3-oxo-3H-xanthen-9-yl)benzoic acid stock solution into 9910 μL buffer (20 mM Tris, 15 mM MgCl₂, pH 7.5). (Stock solution: 10 μM solution of 5-({[2-({[3-({4-[(5-hydroxy-2-methylphenyl)amino]-2-pyrimidinyl}amino)phenyl]carbonyl}amino) ethyl]amino}carbonyl)-2-(6-hydroxy-3-oxo-3H-xanthen-9-yl)benzoic acid in 100% DMSO)

Solution 2 was formed by mixing 53.8 μL of 2.6 μM hTNNI3K with a 6946.2 μL aliquot of Solution 1 (the above 5-({[2-({[3-({4-[(5-hydroxy-2-methylphenyl)amino]-2-pyrimidinyl}amino)phenyl]carbonyl}amino)ethyl]amino}carbonyl)-2-(6-hydroxy-3-oxo-3H-xanthen-9-yl)benzoic acid solution) to make up a 7 mL of mixture of hTNNI3K and 5-({[2-({[3-({4-[(5-hydroxy-2-methylphenyl)amino]-2-pyrimidinyl}amino)phenyl]carbonyl}amino)ethyl]amino}carbonyl)-2-(6-hydroxy-3-oxo-3H-xanthen-9-yl)benzoic acid (Solution 2).

Fifty (50) nL of inhibitors in DMSO (or DMSO controls) were stamped into a 384-well low volume Greiner black plate, followed by addition of 5 μL of Solution 1 to column 18 and 5 μL Solution 2 to columns 1-17 and 19-24 of the plate. The plate was then spun at 500 rpm for 30 seconds and incubated at room temperature for 60 minutes. After that, the fluorescent polarization was measured on Analyst (ex/em: 485/530 nm, Dichroic: 505). For dose response experiments, normalized data were fit by ABASE/XC₅₀ and pXC₅₀=(log((b−y)/(y−a)))/d−log(x), where x is the compound concentration and y is the % activity at specified compound concentration, a is the minimum % activity, b is the maximum % activity, and d is the Hill slope.

The pXC₅₀s are averaged to determine a mean value, for a minimum of 2 experiments. As determined using the above method, the compounds of Example 1-14 exhibited a pXC₅₀ greater than 6.0. For instance, the compounds of Example 5 and Example 11 each inhibited hTNNI3K in the above method with a mean pXC₅₀ of 7.6.

1. A compound according to Formula I or a salt thereof: 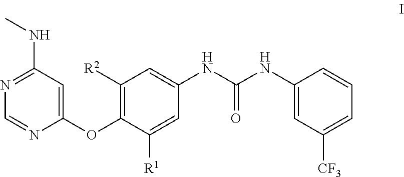
wherein:
R¹ is halogen, C₁-C₄ alkyl, or —ORₐ;
R² is H, halogen, C₁-C₄ alkyl, or —ORₐ; and
Rₐ is C₁-C₄ alkyl which is optionally substituted one to three times by halogen;
provided that when R² is H, R¹ is not methyl.

2. A compound or salt according to claim 1 wherein:
R¹ is selected from chloro, fluoro, bromo, methyl, ethyl, methoxy, and trifluoromethoxy; and
R² is selected from hydrogen, chloro, fluoro, bromo, and methyl.

3. A compound or salt according to claim 1 wherein R² is selected from chloro, fluoro, bromo, and methyl.

4. A pharmaceutical composition comprising the compound or salt according to claim 1 and a pharmaceutically acceptable excipient.

5. A method of inhibiting TNNI3K comprising contacting TNNI3K with the compound or salt according to claim 1.

6. A method for treating congestive heart failure comprising administering to a patient in need thereof an effective amount of the compound or salt according to claim 1.

7. A method for treating congestive heart failure comprising administering to a patient in need thereof the pharmaceutical composition according to claim 4.

8. A compound selected from:
N-(3,5-dichloro-4-{[6-(methylamino)-4-pyrimidinyl]oxy}phenyl)-N′-[3-(trifluoromethyl)phenyl]urea,
N-(3,5-dibromo-4-{[6,-(methylamino)-4-pyrimidinyl]oxy}phenyl)-N′-[3-(trifluoromethyl)phenyl]urea,
N-(3-bromo-5-chloro-4-{[6-(methylamino)-4-pyrimidinyl]oxy}phenyl)-N′-[3-(trifluoromethyl)phenyl]urea,
N-(3,5-difluoro-4-{[6-(methylamino)-4-pyrimidinyl]oxy}phenyl)-N′-[3-(trifluoromethyl)phenyl]urea,
N-(3,5-dimethyl-4-{[6-(methylamino)-4-pyrimidinyl]oxy}phenyl)-N′-[3-(trifluoromethyl)phenyl]urea,
N-[3-bromo-4-{[6-(methylamino)-4-pyrimidinyl]oxy}-5-(methyloxy)phenyl]-N′-[3-(trifluoromethyl)phenyl]urea,
N-(3-chloro-4-{[6-(methylamino)-4-pyrimidinyl]oxy}phenyl)-N′-[3-(trifluoromethyl)phenyl]urea,
N-(3-bromo-4-{[6-(methylamino)-4-pyrimidinyl]oxy}phenyl)-N′-[3-(trifluoromethyl)phenyl]urea,
N-[4-{[6-(methylamino)-4-pyrimidinyl]oxy}-3-(methyloxy)phenyl]-N′-[3-(trifluoromethyl)phenyl]urea,
N-[3-chloro-4-{[6-(methylamino)-4-pyrimidinyl]oxy}-5-(methyloxy)phenyl]-N′-[3-(trifluoromethyl)phenyl]urea,
N-(3-fluoro-4-{[6-(methylamino)-4-pyrimidinyl]oxy}phenyl)-N′-[3-(trifluoromethyl)phenyl]urea,
N-(3-chloro-5-methyl-4-{[6-(methylamino)-4-pyrimidinyl]oxy}phenyl)-N′-[3-(trifluoromethyl)phenyl]urea,
N-(3-chloro-5-ethyl-4-{[6-(methylamino)-4-pyrimidinyl]oxy}phenyl)-N′-[3-(trifluoromethyl)phenyl]urea, and
N-{3-chloro-4-{[6-(methylamino)-4-pyrimidinyl]oxy}-5-[(trifluoromethyl)oxy]phenyl}-N′-[3-(trifluoromethyl)phenyl]urea,
and pharmaceutically acceptable salts thereof.

9. A pharmaceutical composition comprising the compound or salt according to claim 8 and a pharmaceutically acceptable excipient.

10. A method of inhibiting TNNI3K comprising contacting TNNI3K with the compound or salt according to claim 8.

11. A method for treating congestive heart failure comprising administering to a patient in need thereof an effective amount of the compound or salt according to claim 8.

12. A method for treating congestive heart failure comprising administering to a patient in need thereof the pharmaceutical composition according to claim 9.
